SEQUENCE LISTING (4 sequences)

SEQ ID NO: 1 - protein, 286 aa, Homo sapiens
APQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 2 - DNA, 3178 bp, Homo sapiens
gttgcctgtctctaaacccctccacattcccgcggtccttcagactgcccggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaaaaaaaaaaaaaaaaaaaaaaaaaaaaaaaaaaaaaaa

SEQ ID NO: 3 - protein, 285 aa, Homo sapiens
APQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 4 - DNA, 2956 bp, Homo sapiens
ctccacattcccgcggtccttcagactgcccggagagcgcgctctgcctgccgcctgcctgcctgccactgagggttcccagcaccatgagggcctggatcttctttctcctttgcctggccgggagggccttggcagcccctcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattt

(19) United States
(12) Patent Application Publication
(10) Pub. No.: US 2011/0152169 A1
(43) Pub. Date: Jun. 23, 2011

(54) SPARC ANTI-INFLAMMATORY ACTIVITY AND USES THEREOF

(76) Inventors: Kouros Motamed, Irvine, CA (US); Vuong Trieu, Calabasas, CA (US)

(21) Appl. No.: 12/937,925

(22) PCT Filed: Apr. 14, 2009
(86) PCT No.: PCT/US2009/040433

§ 371 Date: Feb. 25, 2011

Related U.S. Application Data
(60) Provisional application No. 61/044,609, filed on Apr. 14, 2008.

(51) Int. Cl. A61K 38/17; A61P 29/00; A61P 25/28; A61P 17/06; A61K 31/7088; A61P 11/06; A61P 9/10; A61P 11/00; A61P 19/08; A61P 19/02; A61P 25/00; A61P 11/14; A61P 11/08; A61P 1/00; A61P 11/02; A61P 19/04; A61P 19/00; A61P 1/16; A61P 13/12

(52) U.S. Cl. 514/1.4; 514/1.1; 514/21.2; 514/16.6; 514/17.9; 514/18.7; 514/44 R; 514/1.7; 514/1.9; 514/1.6; 514/1.5; 514/1.8; 514/16.8

(57) ABSTRACT

The invention provides methods of treating a mammal afflicted with an inflammatory disease, such as peritonitis, peritoneal adhesions or endometriosis, comprising the administration, intraperitoneal or otherwise, of a therapeutically effective amount of a SPARC polypeptide or a SPARC polypeptide-encoding isolated polynucleotide and a pharmacologic carrier.

### CROSS-REFERENCE TO RELATED APPLICATIONS

This application claims the benefit of U.S. Provisional Application No. 61/044,609, filed on Apr. 14, 2008, which is hereby incorporated by reference in its entirety.

### FEDERALLY SPONSORED RESEARCH AND DEVELOPMENT

This invention was made with Government support under Grant Numbers K01-CA089689, HL74279 and HL59699 awarded by the National Institutes of Health. The Government has certain rights in this invention.

### BACKGROUND OF THE INVENTION

Inflammatory diseases and conditions are characterized by excessive inflammation or an excessive immune response. The underlying etiology of an inflammatory disease may be infectious, autoimmune, transplant rejection or other pathologic processes. Some common inflammatory disease include, e.g., peritonitis, plueritis, Rheumatoid arthritis, Inflammatory arthropathies (e.g. ankylosing spondylitis, psoriatic arthritis, Reiter's syndrome), Acute disseminated encephalomyelitis (ADEM), Addison's disease, Ankylosing spondylitis, Autoimmune hepatitis, Autoimmune inner ear disease, Bullous pemphigoid, Coeliac disease, Ulcerative Colitis (one of two types of idiopathic inflammatory bowel disease “IBD”), Crohns Disease (one of two types of idiopathic inflammatory bowel disease “IBD”), Dermatomyositis, Endometriosis, Goodpasture's syndrome, Graves' disease, Guillain-Barré syndrome (GBS), Hashimoto's disease, Kawasaki disease, Interstitial cystitis, Lupus erythematosus, Mixed Connective Tissue Disease, Multiple sclerosis (MS), Myasthenia gravis, Pemphigus Vulgaris, Psoriasis, Psoriatic Arthritis, Polymyositis, Primary biliary chirrosis, Scleroderma, Sjögren's syndrome, Stiff person syndrome, Temporal arteritis (also known as “giant cell arteritis”),Vasculitis, Vitiligo, Wegener's granulomatosis and the like. Although there are numerous anti-inflammatory drugs, there remains a need to develop improved therapies for inflammatory diseases.

Peritonitis is an exemplary inflammatory disease. Peritonitis is an inflammation of the internal lining of the abdominal cavity. The most common causes of peritonitis are bacterial infection and chemical irritation. Bacterial peritonitis is usually secondary to bacterial penetration through an abdominal organ as occurs with disorders such as appendicitis, acute cholecystitis, peptic ulcers, diverticulitis, bowel obstruction, pancreatitis, mesenteric thrombosis, pelvic inflammatory disease, tumor or penetrating trauma, or combinations thereof. In addition, spontaneous bacterial peritonitis (SBP) can develop without an obvious source of contamination, SBP is frequently associated with immunosuppressed states, such as cirrhotic ascites or the nephrotic syndrome. Peritonitis is also a common complication of chronic ambulatory peritoneal dialysis (CAPD).

Although virtually every organism has been implicated in bacterial peritonitis, the most common organisms are E. coli, Pseudomonas aeruginosa, Staphylococcus aureus, C. perfingens, Neisseria gonorrhea, Chlamydia trachomatis, streptococci and enteroococci. The most common fungal agents to cause infectious peritonitis are Candida albicans, Candida parapsilosis, and Aspergillus fumigatus. Non-infectious chemical peritonitis can result from foreign materials introduced into the peritoneum, for example, during surgery or by trauma, Chemical peritonitis can also develop in conditions, such as acute pancreatitis, which introduce irritating endogenous materials, such as digestive enzymes or bile, into the peritoneal cavity.

Peritoneal adhesions and abscesses are frequent long-term complications of peritonitis. Peritoneal adhesions are abnormal fibrous tissue connections between intraperitoneal serosal surfaces. Surgery and abdominal inflammation are the most common causes of peritoneal adhesions. Peritoneal infection is often accompanied by peritoneal inflammation, including exudation of fibrinogen and fibrin into the abdominal cavity. The presence of fibrinogen and fibrin promotes fibrosis and adhesion formation.

Inflammation also results in the intraperitoneal accumulation of growth factors, cytokines, proteases, and extracellular matrix which further promotes fibrosis and adhesion formation. In infectious peritonitis, fibrin deposits may trap infectious agents resulting in abscesses, which in turn can cause more fibrous adhesions. Peritoneal adhesions limit the normal motion and action of the intra-abdominal organs, particularly the normal function of gastrointestinal tract. Peritoneal adhesions also can result in pelvic pain, infertility, and ischemic bowel obstructions.

Peritoneal abscesses are walled-off collections of microorganisms and inflammatory cells and mediators (i.e., “pus”). Peritoneal abscesses are difficult to treat because they are walled-off by fibrous capsules making it difficult to achieve therapeutic levels of antibiotics within peritoneal abscesses. Peritoneal abscesses can be the source of serious infections in distant organs, and sepsis. Thus, peritoneal adhesions and abscesses are a major cause of morbidity and mortality. The effective treatment or prevention of peritonitis can significantly reduce the risk of developing intraperitoneal adhesions and abscesses.

The methods currently used for treating or preventing peritonitis are limited. In some cases, chemical peritonitis can respond to irrigation of the abdominal cavity. The use of multiple re-explorations and intra-operative lavage with large amounts of sterile saline solution has been recommended to decrease the risk of post-operative peritoneal infection, peritonitis and adhesions. However, there is still a significant risk of developing peritonitis and adhesions despite the use of repeated lavages with sterile saline. Various topical antimicrobials have also been tested but none has been widely accepted for source control due to either, lack of efficacy or serious side effects (i.e. sclerosing peritonitis). Further, systemic antibiotic therapy is often required, even if the condition is originally chemical in etiology.

Depending on the type and severity of the peritonitis, the clinical picture could progress to an acute systemic inflammatory response syndrome (SIRS), sepsis or septic shock. The physiopathology of these conditions is complex but it can be associated with the presence of infection and of an acute inflammatory reaction both, locally and systemically. Thus, even in early stages (i.e. SIRS), there is accumulation of pro-inflammatory cytokines in the peritoneal cavity and in the blood that contribute to the establishment of multi-organ failure and death. These cytokines, at least in murine peritonitis models, are mostly derived from activated mast cells in the peritoneal cavity.

Accordingly, there is a need for improved methods of treating or preventing peritonitis. The present invention provides such methods. These and other advantages of the invention, as well as additional inventive features, will be apparent from the description of the invention provided herein.

### SUMMARY OF THE INVENTION

The invention provides methods of treating a mammal afflicted with an inflammatory disease or condition comprising the administration of a therapeutically effective mount of a SPARC polypeptide or a SPARC polypeptide-encoding isolated polynucleotide and a pharmacologic carrier to the afflicted mammal.

The invention provides methods of treating a mammal afflicted with peritonitis comprising the intraperitoneal administration of a therapeutically effective mount of a SPARC polypeptide or a SPARC polypeptide-encoding isolated polynucleotide and a pharmacologic carrier.

The invention provides methods of reducing the incidence of peritoneal adhesions in a mammal comprising the intraperitoneal administration of a therapeutically effective amount of a SPARC polypeptide or a SPARC polypeptide-encoding isolated polynucleotide and a pharmacologic carrier.

The invention provides methods of treating a mammal afflicted with endometriosis comprising the intraperitoneal administration of a therapeutically effective amount of a SPARC polypeptide or a SPARC polypeptide-encoding isolated polynucleotide and a carrier.

### BRIEF DESCRIPTION OF THE DRAWINGS

FIG. 1 depicts the effect of SPARC on the monocyte chemoattractant protein-1 (MCP-1) production and migration of ovarian cancer cells alone or in coculture with mesothelial cells.

FIG. 2 compares the proliferation and invasiveness of ovarian cancer cells expressing or not expressing SPARC, in the presence or absence of anti-MCP-1 antibodies.

FIG. 3 compares the invasiveness and IL-6 production of ovarian cancer cells expressing or not expressing SPARC, alone or in coculture with macrophages, and optionally, peritoneal mesothelial cells.

FIG. 4 depicts the effect of SPARC on the proteolytic activity of ovarian cancer cells and macrophages.

FIG. 5 depicts the prostaglandin E2 (PGE2) production in ovarian cancer cells expressing or not expressing SPARC alone or in coculture with mesothelial cells and/or macrophages, and depicts the effect of PGE2 on the proliferation and invasiveness of ovarian cancer cells expressing or not expressing SPARC.

FIG. 6 depicts the effect of SPARC on the 8-isoprostane production in ovarian cancer cells alone or in coculture with mesothelial cells and/or macrophages.

FIG. 7 compares NF-κB promoter activity in ovarian cancer cells expressing or not expressing SPARC alone or in coculture with macrophages.

### DETAILED DESCRIPTION OF THE INVENTION

DEFINITIONS: In the description that follows, a number of terms are used extensively, the following definitions are provided to facilitate understanding of the invention.

As used herein, a “medicament” is a composition capable of producing an effect that may be administered to a patient or test subject. The effect may be chemical, biological or physical, and the patient or test subject may be human, or a non-human animal, such as a rodent or transgenic mouse. The composition may include small organic or inorganic molecules with distinct molecular composition made synthetically, found in nature, or of partial synthetic origin. Included in this group are nucleotides, nucleic acids, amino acids, peptides, polypeptides, proteins, peptide nucleic acids or complexes comprising at least one of these entities. The medicament may be comprised of the effective composition alone or in combination with a pharmaceutically acceptable excipient.

As used herein, a “pharmaceutically acceptable excipient” includes any and all solvents, dispersion media, coatings, antibacterial, antimicrobial or antifungal agents, isotonic and absorption delaying agents, and the like that are physiologically compatible. The excipient may be suitable for intravenous, intraperitoneal, intramuscular, intrathecal or oral administration. The excipient may include sterile aqueous solutions or dispersions for extemporaneous preparation of sterile injectable solutions or dispersion. Use of such media for preparation of medicaments is known in the art.

As used herein, a “pharmacologically effective amount” of a medicament refers to using an amount of a medicament present in such a concentration to result in a therapeutic level of drug delivered over the term that the drug is used. This may be dependent on the mode of delivery, time period of the dosage, age, weight, general health, sex and diet of the subject receiving the medicament. The determination of what dose is a “pharmacologically effective amount” requires routine optimization, which is within the capabilities of one of ordinary skill in the art.

As used herein, the term “inflammatory disease or condition” refers to any disease or condition is characterized by inflammation or an excessive immune response. The underlying eitology of an inflammatory disease may be infectious, autoimmune, tranplant rejection or other pathologic processes. For example, without limitation, some common inflammatory disease include: peritonitis, plueritis, Rheumatoid arthritis, Inflammatory arthropathies (e.g. ankylosing spondylitis, psoriatic arthritis, Reiter's syndrome), Acute disseminated encephalomyelitis (ADEM), Addison's disease, Ankylosing spondylitis, Autoimmune hepatitis, Autoimmune inner ear disease, Bullous pemphigoid, Coeliac disease, Crohns Disease (one of two types of idiopathic inflammatory bowel disease “IBD”), Dermatomyositis, Endometriosis, Goodpasture's syndrome, Graves' disease, Guillain-Barré syndrome (GBS), Hashimoto's disease, Kawasaki disease, Interstitial cystitis, Lupus erythematosus, Mixed Connective Tissue Disease, Multiple sclerosis (MS), Myasthenia gravis, Pemphigus Vulgaris, Psoriasis, Psoriatic Arthritis, Polymyositis, Primary biliary chirrosis, Scleroderma, Sjögren's syndrome, Stiff person syndrome, Temporal arteritis (also known as “giant cell arteritis”), Ulcerative Colitis (one of two types of idiopathic inflammatory bowel disease “IBD”), Vasculitis, Vitiligo, and Wegener's granulomatosis.

As used herein, a “anti-inflammatory drug” is drug which reduces inflammation. For example, this desirably manifest itself in a reduction in indicators of inflammation, e.g., without limitation, CRP levels, sedimentation rate, white blood cell count, specific antibody levels, inflammatory cells at a disease site, and the like by about 10% or more, for example, about 30%, about 40%, about 50%, about 60%, about 70%, about 80%, or more, to about 2-fold, about 3-fold, about 4-fold, about 5-fold, about 10-fold, about 15-fold, about 20-fold or more. Monitoring of a response may be accomplished by numerous pathological, clinical and imaging methods as described herein and known to persons of skill in the art.

Exemplary anti-inflammatory drugs include glucocorticosteroids, immunosupressive agents including cyclosporine and tacrolimus, the cytotoxic purine antimetabolite azathioprine, alkylating agents, cyclophosphamide, methotrexate, cytarabine, dactinomycin, and thioguanine. Nonsteroidal antiimflammatory drugs include, without limitation, Aspirin (Anacin, Ascriptin, Bayer, Bufferin, Ecotrin, Excedrin), Choline and magnesium salicylates (CMT, Tricosal, Trilisate), Choline salicylate (Arthropan), Celecoxib (Celebrex), Diclofenac potassium (Cataflam), Diclofenac sodium (Voltaren, Voltaren XR), Diclofenac sodium with misoprostol (Arthrotec), Diflunisal (Dolobid), Etodolac (Lodine, Lodine XL), Fenoprofen calcium (Nalfon), Flurbiprofen (Ansaid), Ibuprofen (Advil, Motrin, Motrin IB, Nuprin), Indomethacin (Indocin, Indocin SR), Ketoprofen (Actron, Orudis, Orudis KT, Oruvail), Magnesium salicylate (Arthritab, Mobidin, Mobogesic), Meclofenamate sodium (Meclomen), Mefenamic acid (Ponstel), Meloxicam (Mobic), Nabumetone (Relafen), Naproxen (Naprosyn, Naprelan), Naproxen sodium (Aleve, Anaprox), Oxaprozin (Daypro), Piroxicam (Feldene), Rofecoxib (Vioxx), Salsalate (Amigesic, Anaflex 750, Disalcid, Marthritic, Mono-Gesic, Salflex, Salsitab), Sodium salicylate (various generics), Sulindac (Clinoril) Tolmetin sodium (Tolectin), and Valdecoxib (Bextra). Monoclonal antibodies against the T cell receptor (muromonab-CD3) and interleukin 2 receptors (basiliximab and daclizumab) are also anti-inflammatory.

As used herein, the term “alternative therapeutic regimen” or “alternative therapy” may include for example, biologic response modifiers (including polypeptide-, carbohydrate-, and lipid-biologic response modifiers), toxins, lectins, antiangiogenic agents, and the like. In particular, suitable alternative therapeutic regimens include, For example, such an antibody fragment may be a complete or partial Fc domain.

“Reducing the incidence of peritoneal adhesions” refers to a statically significant reduction in the incidence of adhesions in a clinical trial or an appropriate animal model system.

By “antibodies” it is meant without limitation, monoclonal antibodies, polyclonal antibodies, dimers, multimers, multispecific antibodies (e.g., bispecific antibodies). Antibodies may be murine, human, humanized, chimeric, or derived from other species. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody.

An antibody includes a full-length immunoglobulin molecule or an immunologically active portion of a full-length immunoglobulin molecule, i.e., a molecule that contains an antigen binding site that immunospecifically binds an antigen of a target of interest or part thereof Targets include, cancer cells or other cells that produce autoimmune antibodies associated with an autoimmune disease.

The immunoglobulins disclosed herein can be of any class (e.g., IgG, IgE, IgM, IgD, and IgA) or subclass (e.g., IgG1, IgG2, IgG3, IgG4, IgA1 and IgA2) of immunoglobulin molecule. The immunoglobulins can be derived from any species.

“Antibody fragments” comprise a portion of a full length antibody, which maintain the desired biological activity. “Antibody fragments” are generally the antigen binding or variable region thereof. Examples of antibody fragments include Fab, Fab′, F(ab′)2, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The monoclonal antibodies referenced herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567). Chimeric antibodies of interest herein include “primatized” antibodies comprising variable domain antigen-binding sequences derived from a non-human primate (e.g., Old World Monkey or Ape) and human constant region sequences.

As used herein, a “sensitizer” is a medicament that may enhance the therapeutic effect of an anti-inflammatory drug. A composition or a method of treatment may sensitize to an anti-inflammatory drug if causes an increase in treatment sensitivity by about 10% or more, for example, about 30%, about 40%, about 50%, about 60%, about 70%, about 80%, or more, to about 2-fold, about 3-fold, about 4-fold, about 5-fold, about 10-fold, about 15-fold, about 20-fold or more, compared to treatment sensitivity or resistance in the absence of such composition or method. The determination of sensitivity or resistance to a therapeutic treatment is routine in the art and within the skill of a person versed in the art.

The terms ‘peptide,” “polypeptide,” and “protein” may be used interchangeably, and refer to a compound comprised of at least two amino acid residues covalently linked by peptide bonds or modified peptide bonds, for example peptide isosteres (modified peptide bonds) that may provide additional desired properties to the peptide, such as increased half-life. A peptide may comprise at least two amino acids.

As used herein, the term “polynucleotide” includes RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

As used herein, the term “vector” refers to a polynucleotide compound used for introducing exogenous or endogenous polynucleotide into host cells. A vector comprises a nucleotide sequence, which may encode one or more polypeptide molecules. Plasmids, cosmids, viruses and bacteriophages, in a natural state or which have undergone recombinant engineering, are non-limiting examples of commonly used vectors to provide recombinant vectors comprising at least one desired isolated polynucleotide molecule.

As used herein, a “carrier” or a “pharmacologic carrier” (which are interchangable) refer to any substance suitable as a vehicle for delivering an Active Pharmaceutical Ingredient (API) to a suitable in vitro or in vivo site of action. As such, carriers can act as an excipient for formulation of a therapeutic or experimental reagent containing an API. Preferred carriers are capable of maintaining an API in a form that is capable of interacting with a T cell. Examples of such carriers include, but are not limited to water, phosphate buffered saline, saline, Ringer's solution, dextrose solution, serum-containing solutions, Hank's solution and other aqueous physiologically balanced solutions or cell culture medium. Aqueous carriers can also contain suitable auxiliary substances required to approximate the physiological conditions of the recipient, for example, enhancement of chemical stability and isotonicity. Suitable auxiliary substances include, for example, sodium acetate, sodium chloride, sodium lactate, potassium chloride, calcium chloride, sorbitan monolaurate, triethanolamine oleate, and other substances used to produce phosphate buffer, Tris buffer, and bicarbonate buffer.

SPARC as an Anti-Inflammatory Agent

Secreted protein acidic and rich in cysteine (also known as osteonectin, BM40, or SPARC) (hereafter “SPARC”), is a matrix-associated protein that elicits changes in cell shape, inhibits cell-cycle progression, and influences the synthesis of extracellular matrix (Bradshaw et al., Proc. Nat. Acad. Sci. USA 100: 6045-6050 (2003)). The murine SPARC gene was cloned in 1986 (Mason et al., EMBO J. 5: 1465-1472 (1986)) and a full-length human SPARC cDNA was cloned and sequenced in 1987 (Swaroop et al., Genomics 2: 37-47 (1988)). SPARC expression is developmentally regulated, and is predominantly expressed in tissues undergoing remodeling during normal development or in response to injury. For example, high levels of SPARC protein are expressed in developing bones and teeth (see, e.g., Lane et al., FASEB J., 8, 163 173 (1994); Yan & Sage, J. Histochem. Cytochem. 47:1495-1505 (1999)).

The full-length SPARC protein has multiple functional domains, including an N-terminal acidic domain, a follistatin-like domain that may inhibit cell proliferation and a C-terminal extracellular domain that binds calcium ions with high affinity and inhibits cell proliferation. SPARC has affinity for a wide variety of ligands including cations (e.g., Ca 2+, Cu 2+, Fe 2+), growth factors (e.g., platelet derived growth factor (PDGF), and vascular endothelial growth factor (VEGF)), extracellular matrix (ECM) proteins (e.g., collagen I V and collagen IX, vitronectin, and thrombospondin 1), endothelial cells, platelets, albumin, and hydroxyapaptite (see, e.g., Lane et al., FASEB J., 8, 163 173 (1994); Yan & Sage, J. Histochem. Cytochem. 47:1495-1505 (1999)). SPARC is also known to bind albumin (see, e.g., Schnitzer, J. Biol. Chem., 269, 6072 (1994)).

The inventors have found surprisingly that SPARC has anti-inflammatory activity. While not desiring to be bound by any specific theories, the inventors have found that in a model intraperitoneal system and in vitro SPARC significantly reduces the level marophage chemoattractant protein-1 and inhibits its macrophage chemotactic effect. Overexpression of SPARC in cells also significantly attenuates the macrophage- and mesothelial cell-induced production and activity of interleukin-6, prostanoids (prostaglandins E2 and 8-isoprostanes) as well as matrix metalloproteinases and urokinase plasminogen activator. These effects of SPARC are mediated, in part, through inhibition of nuclear factor-kappaB promoter activation. These results indicate that SPARC can decrease recruitment of macrophages and downregulate the associated inflammation.

The invention provides methods of treating a mammal afflicted with an inflammatory disease comprising the administration of a therapeutically effective mount of a SPARC polypeptide and a pharmacologic carrier to the afflicted mammal, including wherein the SPARC polypeptide is SEQ ID NO: 1 or fragments thereof.

Suitable inflammatory diseases and conditions for treatment in accordance with the invention include., e.g., atherosclerosis, rheumatoid arthritis, sepsis, acute, allergic, or chronic bronchitis, chronic obstructive bronchitis (COPD), coughing, pulmonary emphysema, allergic or non-allergic rhinitis or sinusitis, chronic rhinitis or sinusitis, asthma, alveolitis, Farmer's disease, hyperreactive airways, infectious bronchitis or pneumonitis, pediatric asthma, bronchiectases, pulmonary fibrosis, ARDS (acute adult respiratory distress syndrome), bronchial edema, pulmonary edema, bronchitis, pneumonia or interstitial pneumonia triggered by various causes, such as aspiration, inhalation of toxic gases, or bronchitis, pneumonia or interstitial pneumonia as a result of heart failure, irradiation, chemotherapy, cystic fibrosis, or mucoviscidosis, or alphal-antitrypsin deficiency, acute or chronic inflammatory changes in gall bladder inflammation, Crohn's disease, ulcerative colitis, inflammatory pseudopolyps, juvenile polyps, colitis cystica profunda, pneumatosis cystoides intestinales, diseases of the bile duct, and gall bladder, e.g., gallstones and conglomerates, for the treatment of inflammatory diseases of the joints such as rheumatoid arthritis or inflammatory diseases of the skin and eyes.

Particularly suitable disease and conditions for treatment in accordance with the invention include those involving the degradation of extra-cellular matrix include, but are not limited to, psoriatic arthritis, juvenile arthritis, early arthritis, reactive arthritis, osteoarthritis, ankylosing spondylitis, osteoporosis, muskuloskeletal diseases like tendonitis and periodontal disease, cancer metastasis, airway diseases (COPD, asthma), renal and liver fibrosis, cardiovascular diseases like atherosclerosis and heart failure, and neurological diseases like neuroinflammation, multiple sclerosis, and diseases involving primarily joint degeneration include, but are not limited to, psoriatic arthritis, juvenile arthritis, early arthritis, reactive arthritis, anad osteoarthritis.

Methods for treating inflammatory diseases in accordance with the invention include, for example, methods further comprising the administration of an anti-inflammatory drug and/or further comprising the administration of an antimicrobial agent, such as an antifungal, antiviral or antibiotic.

The invention provides methods of treating a mammal afflicted with peritonitis comprising the intraperitoneal administration of a therapeutically effective mount of a SPARC polypeptide and a pharmacologic carrier, including wherein the SPARC polypeptide is SEQ ID NO: 1 or fragments thereof. Methods in accordance with the invention include, for example, methods further comprising the administration of an anti-inflammatory drug and/or further comprising the administration of an antimicrobial agent, such as an antifungal, antiviral or antibiotic. Suitable causes of peritonitis for treaetment of peritonitis in accordance with the invention includes, e.g., spontaneous bacterial peritonitis, chemical peritonitis, appendicitis, intestinal infract, pancreatitis, gastric rupture, perforating gastric ulcer or truma. Methods for treating peritonitis in accordance with the invention include, for example, methods further comprising the administration of an anti-inflammatory drug and/or further comprising the administration of an antimicrobial agent, such as an antifungal, antiviral or antibiotic.

The invention provides methods of reducing the incidence of peritoneal adhesions in a mammal comprising the intraperitoneal administration of a therapeutically effective amount of a SPARC polypeptide and a pharmacologic carrier, including wherein the SPARC polypeptide is SEQ ID NO: 1 or fragments thereof. Suitable cause of adhensions include, e.g., trauma abdominal or pelvic surgery, and peritonitis.

The invention provides methods of treating a mammal afflicted with endometriosis comprising the intraperitoneal administration of a therapeutically effective amount of a SPARC polypeptide and a carrier, including wherein the SPARC polypeptide is SEQ ID NO: 1 or fragments thereof. Methods of treatment of endometriosis in accordance with the invention include, for example, methods further comprising the administration of an anti-inflammatory drug and/or further comprising the administration of an antimicrobial agent, such as an antifungal, antiviral or antibiotic.

Nucleic Acids for the Expression of SPARC

A SPARC polypeptide can be expressed and purified from a recombinant host cell comprising a polynucleotide encoding a SPARC polypeptide, such as SEQ ID NO: 2. Recombinant host cells may be prokaryotic or eukaryotic, including but not limited to bacteria such as E. coli, fungal cells such as yeast, insect cells including but, not limited to, drosophila and silkworm derived cell lines, and mammalian cells and cell lines.

The invention further provides nucleic acid constructs comprising control elements and a nucleic acid molecule described herein operatively linked to the control elements (e.g., a suitable promoter) for expression of a polypeptide or a polypeptide herein described. Protein expression is dependent on the level of RNA transcription, which is in turn regulated by DNA signals. Similarly, translation of mRNA requires, at the very least, an AUG initiation codon, which is usually located within about 10 to about 100 nucleotides of the 5’ end of the message. Sequences flanking the AUG initiator codon have been shown to influence its recognition by eukaryotic ribosomes, with conformity to a perfect Kozak consensus sequence resulting in optimal translation (see, e.g., Kozak, J. Molec. Biol. 196: 947-950 (1987)). Also, successful expression of an exogenous nucleic acid in a cell can require post- translational modification of a resultant protein. Accordingly, the invention provides plasmids encoding polypeptides wherein the vector is, e.g., pCDNA3.1 or a derivative thereof.

The nucleic acid molecules described herein preferably comprise a coding region operatively linked to a suitable promoter, which promoter is preferably functional in eukaryotic cells. Viral promoters, such as, without limitation, the RSV promoter and the adenovirus major late promoter can be used in the invention. Suitable non- viral promoters include, but are not limited to, the phosphoglycerokinase (PGK) promoter and the elongation factor la promoter. Non-viral promoters are desirably human promoters. Additional suitable genetic elements, many of which are known in the art, also can be ligated to, attached to, or inserted into the inventive nucleic acid and constructs to provide additional functions, level of expression, or pattern of expression. The native promoters for expression of the SPARC family genes also can be used, in which event they are preferably not used in the chromosome naturally encoding them unless modified by a process that substantially changes that chromosome. Such substantially changed chromosomes can include chromosomes transfected and altered by a retroviral vector or similar process. Alternatively, such substantially changed chromosomes can comprise an artificial chromosome such as a HAC, YAC, or BAC.

In addition, the nucleic acid molecules described herein may be operatively linked to enhancers to facilitate transcription. Enhancers are cis-acting elements of DNA that stimulate the transcription of adjacent genes. Examples of enhancers which confer a high level of transcription on linked genes in a number of different cell types from many species include, without limitation, the enhancers from SV40 and the RSV- LTR. Such enhancers can be combined with other enhancers which have cell type-specific effects, or any enhancer may be used alone.

To optimize protein production the inventive nucleic acid molecule can further comprise a polyadenylation site following the coding region of the nucleic acid molecule. Also, preferably all the proper transcription signals (and translation signals, where appropriate) will be correctly arranged such that the exogenous nucleic acid will be properly expressed in the cells into which it is introduced. If desired, the exogenous nucleic acid also can incorporate splice sites (i.e., splice acceptor and splice donor sites) to facilitate mRNA production while maintaining an inframe, full length transcript. Moreover, the inventive nucleic acid molecules can further comprise the appropriate sequences for processing, secretion, intracellular localization, and the like.

The nucleic acid molecules can be inserted into any suitable vector. Suitable vectors include, without limitation, viral vectors. Suitable viral vectors include, without limitation, retroviral vectors, alphaviral, vaccinial, adenoviral, adenoassociated viral, herpes viral, and fowl pox viral vectors. The vectors preferably have a native or engineered capacity to transform eukaryotic cells, e.g., CHO-K1 cells. Additionally, the vectors useful in the context of the invention can be “naked” nucleic acid vectors (i.e., vectors having little or no proteins, sugars, and/or lipids encapsulating them) such as plasmids or episomes, or the vectors can be complexed with other molecules. Other molecules that can be suitably combined with the inventive nucleic acids include without limitation viral coats, cationic lipids, liposomes, polyamines, gold particles, and targeting moieties such as ligands, receptors, or antibodies that target cellular molecules.

One measure of “correspondence” of nucleic acids, peptides or proteins for use herein with reference to the above described nucleic acids and proteins is relative “identity” between sequences, hi the case of peptides or proteins, or in the case of nucleic acids defined according to a encoded peptide or protein correspondence includes a peptide having at least about 50% identity, alternatively at least about 70% identity, alternatively at least about 90% identity, or even about 95% and may also be at least about 98-99% identity to a specified peptide or protein. Preferred measures of identity as between nucleic acids is the same as specified above for peptides with at least about 90% or at least about 98-99% identity being most preferred.

The term “identity” as used herein refers to the measure of the identity of sequence between two peptides or between two nucleic acids molecules. Identity can be determined by comparing a position in each sequence, which may be a line for purposes of comparison. Two amino acid or nucleic acid sequences are considered substantially identical if they share at least about 75% sequence identity, preferably at least about 90% sequence identity and even more preferably at least 95% sequence identity and most preferably at least about 98-99% identity.

Sequence identity may be determined by the BLAST algorithm currently is use and which was originally described in Altschul et al. (1990) J. MoI. Biol. 215:403-410. The BLAST algorithm may be used with the published default settings. When a position in the compared sequence is occupied by the same base or amino acid, the molecules are considered to have shared identity at that position. The degree of identity between sequences is a function of the number of matching positions shared by the sequences.

An alternate measure of identity of nucleic acid sequences is to determine whether two sequences hybridize to each other under low stringency, and preferably high stringency conditions. Such sequences are substantially identical when they will hybridize under high stringency conditions. Hybridization to filter-bound sequences under low stringency conditions may, for example, be performed in 0.5 M NaHPO4, 7% sodium dodecyl sulfate (SDS), 1 mM EDTA at 65° C., and washing in 0.2×SSC/0.1 SDS at 42° C. (see Ausubel et al. (eds.) 1989, Current Protocols in Molecular Biology, Vol. 1, Green Publishing Associates, Inc., and John Wiley & sons, Inc., New York, at p. 2.10.3). Alternatively, hybridization to filter-bound sequences under high stringency conditions, may for example, be performed in 0.5 M NaHPO4, 7% (SDS), 1 mM EDTA at 650 C, and washing in 0.2×SSC/0.1 SDS at 68° C. (see Ausubel et al. (eds.) 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of Principles in Hybridization and the Strategy of Nucleic Acid Probe Assays”, Elsevier, N.Y.). Generally, stringent conditions are selected to be about 50° C. lower than the thermal melting point for the specific sequence at a defined ionic strength and pH.

Those of ordinary skill in the art will recognize that, because of the universality of the genetic code, the knowledge of any given amino acid sequence allows those of ordinary skill in the art to readily envision a finite number of specific polynucleotide sequences that can encode a polypeptide of said amino acid sequence. Further, the ordinarily skilled artisan can readily determine the optimal polynucleotide sequence to encode a polypeptide of said amino acid sequence for expression in any given species via the process of “codon optimization,” which is well know in the art (see, e.g., VILLALOBOS et al.: Gene Designer: a synthetic biology tool for constructing artificial DNA segments. BMC Bioinformatics. 2006 Jun. 6; 7:285).

In addition, the invention provides for an isolated nucleic acid molecule encoding a SPARC polypeptide wherein the isolated nucleic acid molecule hybridizes to SEQ ID NO: 3 under low stringency conditions, preferably under moderately stringent conditions, even more preferably under highly stringent conditions. “High stringency conditions” preferably allow for from about 25% to about 5% mismatch, more preferably from about 15% to about 5% mismatch, and most preferably from about 10% to about 5% mismatch of the nucleic acid sequence. “Moderately stringent conditions” preferably allow for from about 40% to about 15% mismatch, more preferably from about 30% to about 15% mismatch, and most preferably from about 20% to about 15% mismatch of the nucleic acid sequence. “Low stringency conditions” preferably allow for from about 60% to about 35% mismatch, more preferably from about 50% to about 35% mismatch, and most preferably from about 40% to about 35% mismatch of the nucleic acid sequence.

The absence of non-specific binding may be tested by the use of a second target which lacks even a partial degree of complementarity (e.g., less than about 30% identity); in the absence of non-specific binding the probe will not hybridize to the second non-complementary target. There can be partial homology or complete homology (i.e., identity). A partially complementary sequence is one that at least partially inhibits a completely complementary sequence from hybridizing to a target nucleic acid. The inhibition of hybridization of the completely complementary sequence to the target sequence may be examined using a hybridization assay (Southern or Northern blot, solution hybridization and the like) under conditions of low stringency. A “substantially homologous” sequence or probe will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The term “homology” refers to a degree of complementarity.

To optimize protein production the inventive nucleic acid molecule can further comprise a polyadenylation site following the coding region of the nucleic acid molecule. Also, preferably all the proper transcription signals (and translation signals, where appropriate) will be correctly arranged such that the exogenous nucleic acid will be properly expressed in the cells into which it is introduced. If desired, the exogenous nucleic acid also can incorporate splice sites (i.e., splice acceptor and splice donor sites) to facilitate mRNA production while maintaining an inframe, full length transcript. Moreover, the inventive nucleic acid molecules can further comprise the appropriate sequences for processing, secretion, intracellular localization, and the like.

The nucleic acid molecules can be inserted into any suitable vector. Suitable vectors include, without limitation, viral vectors. Suitable viral vectors include, without limitation, retroviral vectors, alphaviral, vaccinial, adenoviral, adenoassociated viral, herpes viral, and fowl pox viral vectors. The vectors preferably have a native or engineered capacity to transform eukaryotic cells, e.g., CHO-K1 cells. Additionally, the vectors useful in the context of the invention can be “naked” nucleic acid vectors (i.e., vectors having little or no proteins, sugars, and/or lipids encapsulating them) such as plasmids or episomes, or the vectors can be complexed with other molecules. Other molecules that can be suitably combined with the inventive nucleic acids include without limitation viral coats, cationic lipids, liposomes, polyamines, gold particles, and targeting moieties such as ligands, receptors, or antibodies that target cellular molecules.

Therefore, the invention provides for a cell transformed or transfected with an inventive nucleic acid molecule described herein. Means of transforming, or transfecting, cells with exogenous DNA molecules are well known in the art. For example, without limitation, a DNA molecule is introduced into a cell using standard transformation or transfection techniques well known in the art such as calcium-phosphate or DEAE-dextran-mediated transfection, protoblast fusion, electroporation, liposomes and direct microinjection (see, e.g., Sambrook & Russell, Molecular Cloning: A Laboratory Manual, Cold Spring Harbor Laboratory Press, New York (2001), pp. 1.1-1.162, 15.1-15.53, 16.1-16.54). A widely used method for transformation is transfection mediated by either calcium phosphate or DEAE-dextran. Depending on the cell type, up to 20% of a population of cultured cells can be transfected at any one time.

Another example of a transformation method is the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells, and the plasmid DNA is transferred to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA randomly integrated into the host chromosome.

Electroporation, the application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of clones genes and for establishment of cell lines that carry integrated copies of the gene of interest.

Liposome transformation involves encapsulation of DNA and RNA within liposomes, followed by fusion of the liposomes with the cell membrane. In addition, DNA that is coated with a synthetic cationic lipid can be introduced into cells by fusion. Alternatively, linear and/or branched polyethylenimine (PEI) can be used in transfection.

Direct microinjection of a DNA molecule into nuclei has the advantage of not exposing the DNA molecule to cellular compartments such as low-pH endosomes. Microinjection is, therefore, used primarily as a method to establish lines of cells that carry integrated copies of the DNA of interest.

Such techniques can be used for both stable and transient tranformation of eukaryotic cells. The isolation of stably transformed cells requires the introduction of a selectable marker in conjunction with the transformation with the gene of interest. Such selectable markers include genes which confer resistance to neomycin as well as the HPRT gene in HPRT negative cells. Selection can require prolonged culture in selection media, at least for about 2-7 days, preferable for at least about 1-5 weeks (see, e.g., Sambrook & Russell, Molecular Cloning: A Laboratory Manual, Cold Spring Harbor Laboratory Press, New York (2001), pp. 16.1-16.54).

Nucleic acid sequences for use in the present invention may also be produced in part or in total by chemical synthesis, e.g. by the phosphoramidite method described by Beaucage, et al. (Tetra. Letts. 22: 1859-1862 (1987)), or the triester method (Matteucci et al., J. Am. Chem. Soc. 103: 3185 (1981)), which may be performed on commercial automated oligonucleotide synthesizers. A double-stranded fragment may be obtained from the single-stranded product of chemical synthesis either by synthesizing the complementary strand and annealing the strand together under appropriate conditions, or by synthesizing the complementary strand using DNA polymerase with an appropriate primer sequence.

Expressing and Purifying the SPARC Protein

In certain embodiments, when expressing and purifying a SPARC polypeptide, techniques for improving protein solubility are employed to prevent the formation of inclusion body (which are insoluble fractions), and therefore obtaining large quantities of the polypeptide. SPARC accumulated in inclusion bodies is an inactive-type SPARC not retaining its physiological activities.

Solubility of a purified SPARC polypeptide can be improved by methods known in the art. For example, solubility may also be improved by expressing a functional fragment, but not the full length SPARC polypeptide. In addition, to increase the solubility of an expressed protein (e.g., in E. coli), one can reduce the rate of protein synthesis by lowering the growth temperature, using a weaker promoter, using a lower copy number plasmid, lowering the inducer concentration, changing the growth medium as described in Georgiou & Valax (Current Opinion Biotechnol. 7:190-197 (1996)). This decreases the rate of protein synthesis and usually more soluble protein is obtained. One can also add prostethic groups or co-factors which are essential for proper folding or for protein stability, or add buffer to control pH fluctuation in the medium during growth, or add 1% glucose to repress induction of the lac promoter by lactose, which is present in most rich media (such as LB, 2xYT). Polyols (e.g., sorbitol) and sucrose may also be added to the media because the increase in osmotic pressure caused by these additions leads to the accumulation of osmoprotectants in the cell, which stabilize the native protein structure. Ethanol, low molecular weight thiols and disulfides, and NaC1 may be added. In addition, chaperones and/or foldases may be co-expressed with the desired polypeptide. Molecular chaperones promote the proper isomerization and cellular targeting by transiently interacting with folding intermediates. E. coli chaperone systems include but, are not limited to: GroES-GroEL, DnaK-DnaJ-GrpE, CIpB.

Foldases accelerate rate-limiting steps along the folding pathway. Three types of foldases play an important role: peptidyl prolyl cis/trans isomerases (PPI's), disulfide oxidoreductase (DsbA) and disulfide isomerase (DsbC), protein disulfide isomerase (PDI) which is an eukaryotic protein that catalyzes both protein cysteine oxidation and disulfide bond isomerization. Co-expression of one or more of these proteins with the target protein could lead to higher levels of soluble target protein.

A SPARC polypeptide can be produced as a fusion protein in order to improve its solubility and production. The fusion protein comprises a SPARC polypeptide and a second polypeptide fused together in frame. The second polypeptide may be a fusion partner known in the art to improve the solubility of the polypeptide to which it is fused, for example, NusA, bacterioferritin (BFR), GrpE, thioredoxin (TRX) and glutathione-S-transferase (GST). Novagen Inc. (Madison, Wis.) provides the pET 43.1 vector series which permit the formation of a NusA-target fusion. DsbA and DsbC have also shown positive effects on expression levels when used as a fusion partner, therefore can be used to fuse with a SPARC polypeptide for achieving higher solubility.

In one embodiment, a SPARC polypeptide is produced as a fusion polypeptide comprising the SPARC polypeptide and a fusion partner thioredoxin, as described in U.S. Pat. No. 6,387,664, hereby incorporated by reference in its entirety. The thioredoxin-SPARC fusion can be produced in E. coli as an easy-to-formulate, soluble protein in a large quantity without losing the physiological activities. Although U.S. Pat. No. 6,387,664 provides a fusion SPARC protein with SPARC fused to the C-terminus of thioredoxin, it is understood, for the purpose of the present invention, a SPARC polypeptide can be fused either to the N-tenninus or the C-terminus of a second polypeptide, so long as its sensitizing function is retained.

In addition to increase solubility, a fusion protein comprising a SPARC polypeptide can be constructed for the easy detection of the expression of the SPARC polypeptide in a cell. In one embodiment, the second polypeptide which fused to the SPARC polypeptide is a reporter polypeptide. The reporter polypeptide, when served for such detection purpose, does not have to be fused with the SPARC polypeptide. It may be encoded by the same polynucleotide (e.g., a vector) which also encodes the SPARC polypeptide and be co-introduced and co-expressed in a target cell.

Quantitative parameters such as mean fluorescence intensity and variance can be determined from the fluorescence intensity profile of the cell population (Shapiro, H., 1995, Practical Flow Cytometry, 217-228). Non-limiting examples of reporter molecules useful in the invention include luciferase (from firefly or other species), chloramphenicol acetyltransferase, .beta.-galactosidase, green fluorescent protein (GFP), enhanced green fluorescent protein (EGFP), and dsRed.

Expression of the SPARC polypeptide (either by itself, or as a fusion protein) can also be directly determined by an immunoassay such as an ELISA (enzyme-linked immunoabsorbent assay) (see, e.g., U.S. Pat. Nos. 5,962,320; 6,187,307; and 6,194,205), Western blot, or by other methods routine in the art. The expression of a SPARC polypeptide can be indirectly detected by detecting the transcript of the protein (e.g., by hybridization analysis such as Northern blot or DNA Microarray, or by PCR).

In one embodiment, a polynucleotide encoding a second polypeptide is fused to a polynucleotide encoding a SPARC polypeptide through an intervening linker sequence which encodes for a linker polypeptide.

In another embodiment, the linker polypeptide comprises a protease cleavage site comprising a peptide bond which is hydrolyzable by a protease. As a result, the SPARC polypeptide can be separated from the second polypeptide after expression by proteolysis. The linker can comprise one or more additional amino acids on either side of the bond to which the catalytic site of the protease also binds (see, e.g., Schecter & Berger, Biochem. Biophys. Res. Commun. 27, 157-62 (1967)). Alternatively, the cleavage site of the linker can be separate from the recognition site of the protease and the two cleavage site and recognition site can be separated by one or more (e.g., two to four) amino acids. In one aspect, the linker comprises at least about 2, 3, 4, 5, 6, 7, 8, 9, about 10, about 20, about 30, about 40, about 50 or more amino acids. More preferably the linker is from about 5 to about 25 amino acids in length, and most preferably, the linker is from about 8 to about 15 amino acids in length.

Some proteases useful according to the invention are discussed in the following references: Hooper et al., Biochem. J. 321: 265-279 (1997); Werb, Cell 91: 439-442 (1997); Wolfsberg et al., J. Cell Biol. 131: 275-278 (1995); Murakami & Etlinger, Biochem. Biophys. Res. Comm. 146: 1249-1259 (1987); Berg et al., Biochem. J. 307: 313-326 (1995); Smyth and Trapani, Immunology Today 16: 202-206 (1995); Talanian et al., J. Biol. Chem. 272: 9677-9682 (1997); and Thornberry et al., J. Biol. Chem. 272: 17907-17911 (1997). Cell surface proteases also can be used with cleavable linkers according to the invention and include, but are not limited to: Aminopeptidase N; Puromycin sensitive aminopeptidase; Angiotensin converting enzyme; Pyroglutamyl peptidase II; Dipeptidyl peptidase IV; N-arginine dibasic convertase; Endopeptidase 24.15; Endopeptidase 24.16; Amyloid precursor protein secretases alpha, beta and gamma; Angiotensin converting enzyme secretase; TGF alpha secretase; TNF alpha secretase; FAS ligand secretase; TNF receptor-I and -II secretases; CD30 secretase; KL1 and KL2 secretases; IL6 receptor secretase; CD43, CD44 secretase; CD 16-I and CD16-II secretases; L-selectin secretase; Folate receptor secretase; MMP 1,2, 3,7, 8,9, 10,11, 12,13, 14, and 15; Urokinase plasminogen activator; Tissue plasminogen activator; Plasmin; Thrombin; BMP-1 (procollagen C-peptidase); ADAM 1,2, 3,4, 5,6, 7,8, 9,10, and 11; and, Granzymes A, B, C, D, E, F, G, and H.

An alternative to relying on cell-associated proteases is to use a self-cleaving linker. For example, the foot and mouth disease virus (FMDV) 2A protease may be used as a linker. This is a short polypeptide of 17 amino acids that cleaves the polyprotein of FMDV at the 2A/2B junction. The sequence of the FMDV 2A propeptide is NFDLLKLAGDVESNPGP. Cleavage occurs at the C-terminus of the peptide at the final glycine-proline amino acid pair and is independent of the presence of other FMDV sequences and cleaves even in the presence of heterologous sequences.

Insertion of this sequence between two protein coding regions (i.e., between the SPARC polypeptide and the second polypeptide of a fusion protein according to the invention) results in the formation of a self-cleaving chimera which cleaves itself into a C-terminal fragment which carries the C-terminal proline of the 2A protease on its N-terminal end, and an N-terminal fragment that carries the rest of the 2A protease peptide on its C-terminus (see, e.g., de Felipe et al., Gene Therapy 6: 198-208 (1999)). Self-cleaving linkers and additional protease-linker combinations are described further in PCT Publication WO 01/20989, the entirety of which is incorporated by reference herein.

Polynucleotides encoding linker sequences described above can be cloned from sequences encoding the natural substrates of an appropriate protease or can be chemically synthesized using methods routine in the art.

Affinity chromatography employing SPARC ligands and/or anti-SPARC antibodies can also by used to purify SPARC polypeptides in accordance with the invention. Affinity chromatography can be used alone or in conjunction with ion-exchange, molecular sizing, or HPLC chromatographic techniques. Such chromatographic approach can be performed using columns or in batch formats. Such chromatographic purification methods are well known in the art.

The expression of SPARC protein in the sample can be detected and quantified by any suitable method known in the art. Suitable methods of protein detection and quantification include Western blot, enzyme-linked immunosorbent assay (ELISA), silver staining, the BCA assay (Smith et al., Anal. Biochem. 150, 76-85 (1985)), the Lowry protein assay (described in, e.g., Lowry et al., J. Biol. Chem. 193, 265-275 (1951)), which is a calorimetric assay based on protein-copper complexes, and the Bradford protein assay (described in, e.g., Bradford et al., Anal. Biochem. 72, 248 (1976)), which depends upon the change in absorbance in Coomassie Blue G-250 upon protein binding. Tumor biopsy can be analyzed by any of the preceding methods or it can be analyzed by immunohistochemistry using an anti-SPARC antibody, e.g., an anti-SPARC polypeptide anbody, (either monoclonal or polyclonal) in conjunction with appropriate visualization system (i.e., HRP substrate and HRP-conjugated secondary antibody).

Administering the SPARC Protein or Polynucleotide

SPARC proteins will typically be administered with a carrier. The composition can further comprise any other suitable components, especially for enhancing the stability of the composition and/or its end-use. Accordingly, there is a wide variety of suitable formulations of the composition of the invention. The following formulations and methods are merely exemplary and are in no way limiting.

The carrier typically will be liquid, but also can be solid, or a combination of liquid and solid components. The carrier desirably is physiologically acceptable (e.g., a pharmaceutically or pharmacologically acceptable) carrier (e.g., excipient or diluent). Physiologically acceptable carriers are well known and are readily available. The choice of carrier will be determined, at least in part, by the location of the target tissue and/or cells, and the particular method used to administer the composition.

Typically, such compositions can be prepared as injectables, either as liquid solutions or suspensions; solid forms suitable for using to prepare solutions or suspensions upon the addition of a liquid prior to injection can also be prepared; and the preparations can also be emulsified. The pharmaceutical formulations suitable for injectable use include sterile aqueous solutions or dispersions; formulations containing known protein stabilizers and lyoprotectants, formulations including sesame oil, peanut oil or aqueous propylene glycol, and sterile powders for the extemporaneous preparation of sterile injectable solutions or dispersions. In all cases the formulation must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

The SPARC proein can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The composition can further comprise any other suitable components, especially for enhancing the stability of the composition and/or its end-use. Accordingly, there is a wide variety of suitable formulations of the composition of the invention. The following formulations and methods are merely exemplary and are in no way limiting.

Formulations suitable for administration via inhalation include aerosol formulations. The aerosol formulations can be placed into pressurized acceptable propellants, such as dichlorodifluoromethane, propane, nitrogen, and the like. They also can be formulated as non-pressurized preparations, for delivery from a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described. In this regard, the formulation desirably is suitable for administration at the site of inflammation, but also can be thus formulated for intravenous injection, intraperitoneal injection, subcutaneous injection, and the like.

Formulations suitable for anal administration can be prepared as suppositories by mixing the active ingredient with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

In addition, the composition of the invention can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the pharmaceutical composition and physiological distress.

SPARC polypeptide or SPARC encoding isolated polynucleotide can be administered conjunction with one or more additional therapeutic agents, e.g., one or more compounds selected from the group consisting of anti-infective agents (e.g., anti-bacterial agents (such as, e.g., antibiotics), anti-fungal agents and anti-viral agents), anti-inflammatory agents, recombinant proteins or antibodies, one or more synthetic drugs and combinations thereof Administering such therapeutic agents in conjunction with the SPARC polypeptide or SPARC encoding isolated polynucleotide can include administering one or more of such additional agents, e.g., prior to, during (e.g., contemporaneously, by co-administration or in combination with), or following administration of the ORP water solution.

Suitable antibiotics can include, without limitation, penicillin, cephalosporins or other β-lactams, macrolides (e.g., erythromycin, 6-O-methylerythromycin, and azithromycin), fluoroquinolones, sulfonamides, tetracyclines, aminoglycosides, clindamycin, quinolones, metronidazole, vancomycin, chloramphenicol, antibacterially effective derivatives thereof, and combinations thereof Suitable anti-infective agents also can include antifungal agents such as, for example, amphotericin B, fluconazole, flucytosine, ketoconazole, miconazole, derivatives thereof, and combinations thereof Suitable anti-inflammatory agents can include, e.g., one or more anti-inflammatory drugs, e.g., one or more anti-inflammatory steroids or one or more non-steroidal anti-inflammatory drugs (NSAIDs). Exemplary anti-inflammatory drugs can include, e.g., cyclophilins, FK binding proteins, anti-cytokine antibodies (e.g. anti-TNF), steroids, and NSAIDs.

In accordance with the invention, a SPARC polypeptide or SPARC encoding isolated polynucleotide can be administered alone or in combination with one or more pharmaceutically acceptable carriers, e.g., vehicles, adjuvants, excipients, diluents, combinations thereof, and the like. One skilled in the art can easily determine the appropriate formulation and method for administering the SPARC polypeptide or SPARC encoding isolated polynucleotide used in accordance with the present invention. For instance, the use of a gel based formulation containing the ORP water solution can be used to maintain hydration of the peritoneal cavity while providing a barrier against microorganisms. Suitable gel formulations are described, e.g., in U.S. Patent Application Publication No. US 2005/0142157. Any necessary adjustments in dose can be readily made by a skilled practitioner to address the nature and/or severity of the condition being treated in view of one or more clinically relevant factors, such as, e.g., side effects, changes in the patient's overall condition, and the like.

SPARC Gene Therapy

Gene therapy is a medical intervention that involves modifying the genetic material of living cells to fight disease. Gene therapy is being studied in clinical trials (research studies with humans) for many different types of cancer and for other diseases. Accordingly, the invention further provides for an isolated nucleic acid molecule encoding a SPARC polypeptide suitable for use in “gene therapy” (see, e.g., Patil et al., AAPS J. 7(1):E61-77 (2005)).

Gene therapy can be performed both ex vivo and in vivo. Typically, in ex vivo gene therapy clinical trials, cells from the patient's blood or bone marrow are removed and grown in the laboratory. The cells are exposed to the virus that is carrying the desired gene. The virus enters the cells, and the desired gene becomes part of the cells' DNA. The cells grow in the laboratory and are then returned to the patient by injection into a vein. Using in vivo gene therapy, vectors such as, e.g., viruses or liposomes may be used to deliver the desired gene to cells inside the patient's body.

The invention further provides for an isolated nucleic acid molecule encoding a SPARC polypeptide suitable for use in “gene therapy. Suitable nucleic acids include, but are not limited to nucleic acids comprising SEQ ID NO: 2 or modifications and homologues thereof as described herein. One of the goals of gene therapy is to supply cells with altered genes, such as, e.g., an isolated nucleic acid molecule encoding a SPARC polypeptide. Gene therapy is also being studied as a way to change how a cell functions; for example, by stimulating immune system cells to attack cancer cells.

In general, a gene is delivered to the cell using a “vector” such as those disclosed herein. The most common types of vectors used in gene therapy are viruses. Viruses used as vectors in gene therapy are genetically disabled; they are unable to reproduce themselves. Most gene therapy clinical trials rely on mouse retroviruses to deliver the desired gene. Other viruses used as vectors include adenoviruses, adeno-associated viruses, poxviruses, and the herpes virus. Suitable viral gene therapy vectors and modes of their administration in vivo and ex vivo are know in the art.

Gene therapy can be done both ex vivo and in vivo. Typically, in ex vivo gene therapy clinical trials, cells from the patient's blood or bone marrow are removed and grown in the laboratory. The cells are exposed to the virus that is carrying the desired gene. The virus enters the cells, and the desired gene becomes part of the cells' DNA. The cells grow in the laboratory and are then returned to the patient by injection into a vein. In in vivo gene therapy, vectors such as, e.g., viruses or liposomes are used to deliver the desired gene to cells inside the patient's body.

Modification of SPARC Proteins

The amino acids comprising a peptide or protein described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide.

Examples of modifications to peptides may include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer- RNA mediated addition of amino acids to proteins such as arginylation, and ubiquitination. See, for instance, Proteins-Structure and Molecular Properties, 2nd ed., T. E. Creighton, W. H. Freeman and Company, New York, 1993 and Wold F, Posttranslational Protein Modifications: Perspectives and Prospects, pgs. 1-12 in Posttranslational Covalent Modification of Proteins, B. C. Johnson, ed., Academic Press, New York, 1983; Seifter et ah, Analysis for protein modifications and nonprotein cofactors, Meth. Enzymol. (1990) 182: 626-646 and Rattan et al. (1992), Protein Synthesis: Posttranslational Modifications and Aging, “ Ann NY Acad Sci 663: 48-62.

A substantially similar sequence is an amino acid sequence that differs from a reference sequence only by one or more conservative substitutions as discussed herein. Such a sequence may, for example, be functionally homologous to another substantially similar sequence. It will be appreciated by a person of skill in the art the aspects of the individual amino acids in a peptide of the invention that may be substituted.

Amino acid sequence similarity or identity may be computed by, e.g., using the BLASTP and TBLASTN programs which employ the BLAST (basic local alignment search tool) 2.0 algorithm. Techniques for computing amino acid sequence similarity or identity are well known to those skilled in the art, and the use of the BLAST algorithm is described in ALTSCHUL et al. 1990, J MoI. Biol. 215: 403-410 and ALTSCHUL et al. (1997), Nucleic Acids Res. 25: 3389-3402.

Sequences on which to perform an alignment may be collected from numerous databases. Examples of protein databases include SWISS-PROT, which also provides a high level of annotation relating to the function of a protein, its domains structure, post-translational modifications, variants (Bairoch A. and Apweiler R. (2000) Nucleic Acids Res. 28(1):45-48; Bairoch A. and Apweiler R. (1997) J. MoI. Med. 75(5):312-316; Junker V. L. et al.(1999) Bioinformatics 15(12): 1066-1007), TrEMBL a computer-annotated supplement of SWISS-PROT that contains all the translations of EMBL nucleotide sequence entries (Bairoch A. and Apweiler R. (2000) Nucleic Acids Res. 28(0:45-48) and nr database compares all non-redundant GenBank CDS translations plus protein sequences from other databases such as PDB, SwissProt, PIR and PRF.

Alignments of protein sequences may be conducted using existing algorithms to search databases for sequences similar to a query sequence. One alignment method is the Smith-Waterman algorithm (Smith, T. F. and Waterman, M. S. 1981. Journal of Molecular Biology 147(1):195-197), which is useful in determining how an optimal alignment between the query sequence and a database sequence can be produced. Such an alignment is obtained by determining what transformations the query sequence would need to undergo to match the database sequence. Transformations include substituting one character for another and inserting or deleting a string of characters. A score is assigned for each character-to-character comparison-positive scores for exact matches and some substitutions, negative scores for other substitutions and insertions/deletions. Scores are obtained from statistically-derived scoring matrices. The combination of transformations that results in the highest score is used to generate an alignment between the query sequence and database sequence. The Needleman-Wunsch (Needleman, S. B. and Wunsch, C D. 1970. Journal of Molecular Biology 48(3):443-453) algorithm is similar to the Smith- Waterman algorithm, but sequence comparisons are global, not local. Global comparisons force an alignment of the entire query sequence against the entire database sequence. While local alignments always begin and end with a match, global alignments may begin or end with an insertion or deletion (indel). For a given query sequence and database sequence, a global score will be less than or equal to a local score due to indels on the ends. As an alternative to the above algorithms, a Hidden Markov Model (HMM) search (Eddy, S. R. 1996. Current Opinion in Structural Biology 6(3):361-365) could be used to generate protein sequence alignments. HMM scoring weighs the probability of a match being followed by insertions/deletions or vice-versa. In addition, HMMs allow insertion to deletion transitions (and vice versa) and scoring of begin and end states to control whether a search is run globally or locally.

One or more of the above algorithms may be used in an alignment program to generate protein sequence alignments. A person skilled in the art has numerous sequence alignment programs to choose from, that incorporate a variety of different algorithms. One example of an alignment program is BLASTP (Altschul, S. F., et al. (1997) Nucleic Acids Res. 25(17):3389-3402). Other alignment programs areCLUSTAL W and PILEUP. The standard output from a BLASTP run contains enough information to conduct further indel analysis as described below.

Amino acids may be described as, for example, polar, non-polar, acidic, basic, aromatic or neutral. A polar amino acid is an amino acid that may interact with water by hydrogen bonding at biological or near-neutral pH. The polarity of an amino acid is an indicator of the degree of hydrogen bonding at biological or near- neutral pH. Examples of polar amino acids include serine, proline, threonine, cysteine, asparagine, glutamine, lysine, histidine, arginine, aspartate, tyrosine and glutamate. Examples of non-polar amino acids include glycine, alanine, valine leucine, isoleucine, methionine, phenylalanine, and tryptophan. Acidic amino acids have a net negative charge at a neutral pH. Examples of acidic amino acids include aspartate and glutamate. Basic amino acids have a net positive charge at a neutral pH. Examples of basic amino acids include arginine, lysine and histidine. Aromatic amino acids are generally nonpolar, and may participate in hydrophobic interactions. Examples of aromatic amino acids include phenylalanine, tyrosine and tryptophan. Tyrosine may also participate in hydrogen bonding through the hydroxyl group on the aromatic side chain. Neutral, aliphatic amino acids are generally nonpolar and hydrophobic. Examples of neutral amino acids include alanine, valine, leucine, isoleucine and methionine. An amino acid may be described by more than one descriptive category. Amino acids sharing a common descriptive category may be substitutable for each other in a peptide.

Nomenclature used to describe the peptide compounds of the present invention follows the conventional practice where the amino group is presented to the left and the carboxy group to the right of each amino acid residue. In the sequences representing selected specific embodiments of the present invention, the amino- and carboxy-terminal groups, although not specifically shown, will be understood to be in the form they would assume at physiologic pH values, unless otherwise specified. In the amino acid structure formulae, each residue may be generally represented by a one-letter or three-letter designation, as is known to those of ordinary skill in the art.

The hydropathy index of an amino acid is a scale indicating the tendency of an amino acid to seek out an aqueous environment (negative value) or a hydrophobic environment (positive value) (KYTE & DOOLITTLE 1982. J MoI Biol 157:105-132). Hydropathy indices of the standard amino acids include alanine (1.8), arginine (−4.5), asparagine (−3.5), aspartic acid (−3.5), cysteine (2.5), glutamine (−3.5), glutamic acid (−3.5), glycine (−0.4), histidine (−3.2), isoleucine (4.5), leucine (3.8), lysine (−3.9), methionine (1.9), phenylalanine (2.8), proline (−1.6), serine (−0.8), threonine (−0.7), tryptophan(−0.9), tyrosine (−1.3), and valine (4.2). Amino acids with similar hydropathy indices may be substitutable for each other in a peptide.

Amino acids comprising the peptides described herein will be understood to be in the L- or D- configuration. In peptides and peptidomimetics of the present invention, D-amino acids may be substitutable for L-amino acids.

Amino acids contained within the peptides of the present invention, and particularly at the carboxy-or amino- terminus, may be modified by methylation, amidation, acetylation or substitution with other chemical groups which may change the circulating half-life of the peptide without adversely affecting their biological activity. Additionally, a disulfide linkage may be present or absent in the peptides of the invention.

Nonstandard amino acids may occur in nature, and may or may not be genetically encoded. Examples of genetically encoded nonstandard amino acids include selenocysteine, sometimes incorporated into some proteins at a UGA codon, which may normally be a stop codon, or pyrrolysine, sometimes incorporated into some proteins at a UAG codon, which may normally be a stop codon. Some nonstandard amino acids that are not genetically encoded may result from modification of standard amino acids already incorporated in a peptide, or may be metabolic intermediates or precursors, for example. Examples of nonstandard amino acids include 4-hydroxyproline, 5-hydroxylysine, 6-N-methyllysine, gamma- carboxyglutamate, desmosine, selenocysteine, ornithine, citrulline, lanthionine, 1-aminocyclopropane-1 -carboxylic acid, gamma-aminobutyric acid, carnitine, sarcosine, or N-formylmethionine. Synthetic variants of standard and non-standard amino acids are also known and may include chemically derivatized amino acids, amino acids labeled for identification or tracking, or amino acids with a variety of side groups on the alpha carbon. Examples of such side groups are known in the art and may include aliphatic, single aromatic, polycyclic aromatic, heterocyclic, heteronuclear, amino, alkylamino, carboxyl, carboxamide, carboxyl ester, guanidine, amidine, hydroxyl, alkoxy, mercapto-, alkylmercapto-, or other heteroatom- containing side chains. Other synthetic amino acids may include alpha-imino acids, non-alpha amino acids such as beta-amino acids, des-carboxy or des-amino acids. Synthetic variants of amino acids may be synthesized using general methods known in the art, or may be purchased from commercial suppliers, for example RSP Amino Acids LLC (Shirley, Mass.).

In order to further exemplify what is meant by a conservative amino acid substitution, Groups A-F are listed below. The replacement of one member of the following groups by another member of the same group is considered to be a conservative substitution.

Group A includes leucine, isoleucine, valine, methionine, phenylalanine, serine, cysteine, threonine, and modified amino acids having the following side chains: ethyl, iso-butyl, —CH2CH2OH, —CH2CH2CH2OH, —CH2CHOHCH3 and CH2SCH3.

Group B includes glycine, alanine, valine, serine, cysteine, threonine, and a modified amino acid having an ethyl side chain.

Group C includes phenylalanine, phenylglycine, tyrosine, tryptophan, cyclohexylmethyl, and modified amino residues having substituted benzyl or phenyl side chains.

Group D includes glutamic acid, aspartic acid, a substituted or unsubstituted aliphatic, aromatic or benzylic ester of glutamic or aspartic acid (e.g., methyl, ethyl, n-propyl, iso-propyl, cyclohexyl, benzyl, or substituted benzyl), glutamine, asparagine, CO—NH-alkylated glutamine or asparagine (e.g., methyl, ethyl, n-propyl, and iso-propyl), and modified amino acids having the side chain —(CH2)3COOH, an ester thereof (substituted or unsubstituted aliphatic, aromatic, or benzylic ester), an amide thereof, and a substituted or unsubstituted N-alkylated amide thereof.

Group E includes histidine, lysine, arginine, N-nitroarginine, p-cycloarginine, g-hydroxyarginine, N-amidinocitruline, 2-amino guanidinobutanoic acid, homologs of lysine, homologs of arginine, and ornithine.

Group F includes serine, threonine, cysteine, and modified amino acids having C1-C5 straight or branched alkyl side chains substituted with —OH or —SH.

Groups A-F are exemplary and are not intended to limit the invention.

Such conservative mutations and amino acid changes can be introduced by any suitable method known to those of ordinary skill in the art for site directed mutagenesis.

The following examples demonstrate the effect of SPARC on cancer-associated inflammation in various in vitro model systems which are well known to one of ordinary skill in the art to be reflective of the pathophysiology of ovarian cancer. These examples serve to illustrate the invention but should not be construed as in any way limiting its scope.

### EXAMPLE 1

This example demonstrates that SPARC inhibits MCP-1 production.

Human ovarian carcinoma cell lines (SKOV3 and OVCAR3) and human peritoneal mesothelial cell line (Meso301) were obtained and maintained as described previously (Said et al., Am J Pathol, 170: 1054-63 (2007) and Said et al., Am J Pathol, 167: 1739-52 (2005)). Human monocytoid cell line (U937) was purchased from ATCC (Manassas, Va.) and was maintained in RPMI 1640 containing 10% FBS (Atlanta Biologicals, Norcross, Ga.).

Replication-deficient adenoviruses expressing either SPARC or green fluorescent protein (GFP) under the control of the cytomegalovirus promoter were generated as described previously (Said et al., Am J Pathol, 170: 1054-63 (2007)). Ovarian cancer cell lines were transduced for 24 hours with adenovirus expressing GFP (Ad-GFP) or adenovirus expressing GFP and SPARC (Ad-GFP-SPARC) in complete growth medium at a multiplicity of infection of 15 to 25 as described previously (Said et al., Am J Pathol, 170: 1054-63 (2007)). SPARC protein was detected in cell lysates and conditioned medium of SKOV3 and OVCAR3 ovarian cancer cell lines after Ad-GFP-SPARC transduction, but not after Ad-GFP-transduction.

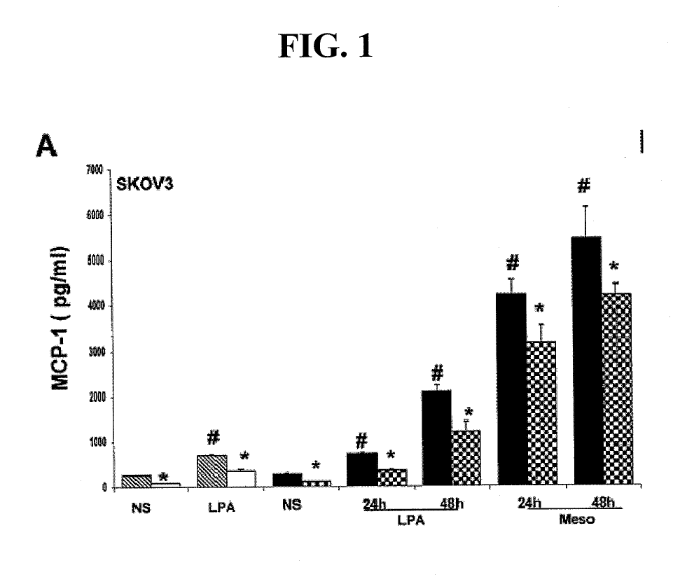

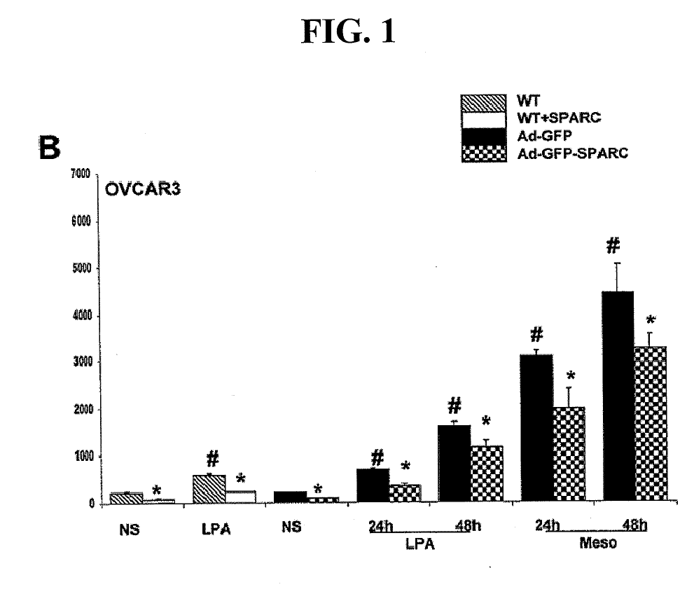

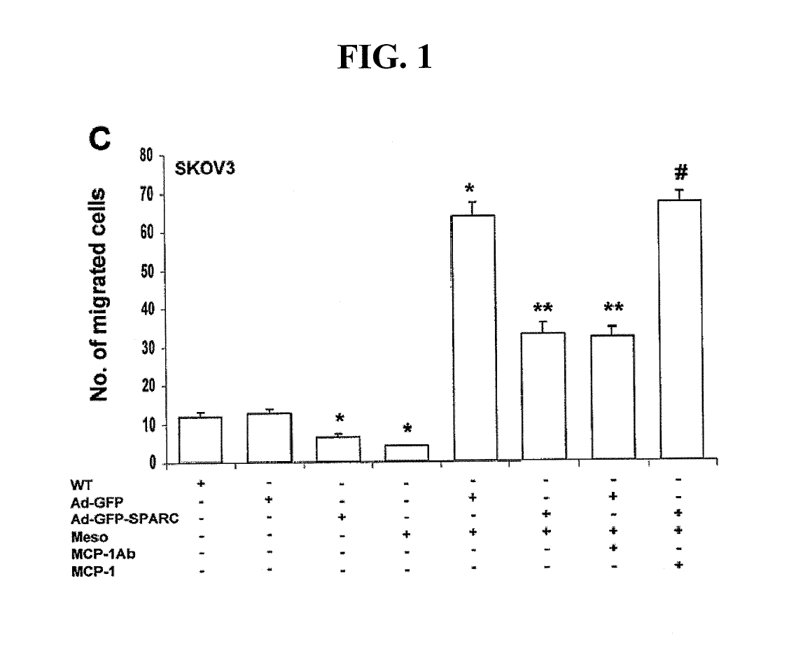

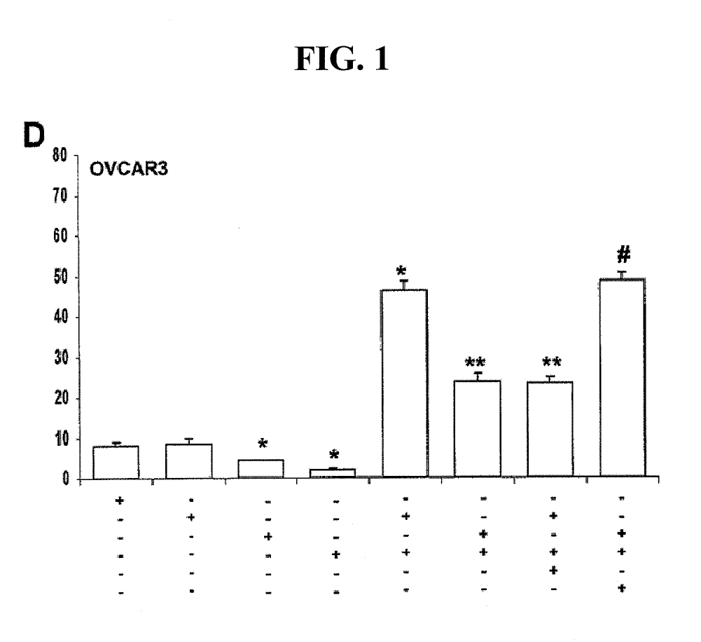

Confluent monolayers (−106 cells, serum starved overnight) of wild type (WT) human ovarian cancer cell lines SKOV3 or OVCAR3, were stimulated with 50 μM lysophosphatidic acid (LPA) for 24 hours in the presence and absence of SPARC (10 μg/ml). Similarly, adenovirus-transduced SKOV3 and OVCAR3 were stimulated with 50 [M LPA for 24 to 48 hours. Alternatively, adenovirus-transduced SKOV3 and OVCAR3 were seeded onto confluent monolayers or cocultured with Meso301 for 24 to 48 hours. MCP-1 protein secreted in the conditioned medium (CM) of SKOV3 and OVCAR3 was determined by ELISA using a commercially available kit (RayBioTech, Inc., Norcross, Ga.). Controls included cells that were not stimulated (NS) with LPA. The results shown /in FIG. 1 are the mean±SEM of a representative of three independent experiments, each performed in duplicate. *P<0.05, compared to matched nonstimulated or Ad-GFP condition. #P<0.05, compared to nonstimulated WT or Ad-GFP cells.

There was a time-dependent increase of MCP-1 production by SKOV3 and OVCAR3 cell lines after LPA stimulation (approximately three- and nine-fold increase after 24 and 48 hours, respectively). Exogenous SPARC, as well as adenoviral expression of SPARC in SKOV3 and OVCAR3 cell lines, significantly inhibited both basal (54% to 64%) and LPA-induced MCP-1 production (54% and 43% inhibition in SKOV3 at 24 and 48 hours, respectively; 59% and 30% inhibition in OVCAR3 at 24 and 48 hours, respectively. Coculture of peritoneal mesothelial cells, Meso301, with ovarian cancer cells further augmented MCP-1 production in a time-dependent manner. The increase was 16- to 21-fold with SKOV3 at 24 and 48 hours, respectively, and 14- to 20-fold with OVCAR3 at 24 and 48 hours, respectively. Overexpression of SPARC significantly attenuated the augmented MCP-1 production in the cocultures by -26% to 36% in SKOV3 at 24 and 48 hours, respectively, and 36% to 28% in OVCAR3 at 24 and 48 hours, respectively. This example demonstrated that MCP-1 secretion by ovarian cancer cells is augmented by LPA stimulation and in cocultures with peritoneal mesothelial cells, and that overexpression of SPARC in ovarian cancer cells attenuates MCP-1 production.

### EXAMPLE 2

This example demonstrates an inhibitory effect of SPARC on the migration of macrophages induced by ovarian cancer cells.

Macrophage chemotaxis assays were performed using 3-μm pore-size polycarbonate inserts (Corning Costar). Ovarian cancer cell transduction was performed as described in Example 1. Twenty four hour-transduced SKOV3 and OVCAR3 cells were seeded at 2×105 cells per well in 600 μl of serum free medium (SFM) in 24-well plates and grown to confluence, or were seeded onto confluent monolayers of Meso301 grown in 24-well plates. U937 macrophages (105 cells in 100 μl SFM) were added to the top chamber of the transwells. In some experiments, neutralizing anti-MCP-1 antibody (25 μg/ml) (Chemicon, Temecula, Calif.) was included in Ad-GFP-transduced ovarian cancer cell-Meso301 cocultures. After incubation at 37° C. for 90 minutes, the contents of the upper chamber were aspirated, washed with phosphate buffered saline (PBS), and stained with DAPI (Sigma). U937 migration was assessed by counting the number of cells attached to the lower surface of the membrane in six high-power fields per well, using a fluorescent microscope equipped with a Q-imaging digital camera (Leica Microsystems, Wetzlar, Germany). The results shown in FIG. 1 are the mean±SEM of a representative of three independent experiments, each performed in triplicate. *P<0.05, compared to nonstimulated (NS) or Ad-GFP-transduced cells. **P<0.05, compared to cocultures of Meso301 and Ad-GFP-transduced ovarian cancer cells.

Monolayers of SKOV3 and OVCAR3 stimulated the migration of U937 macrophages toward the tumor cells. This chemotactic effect was significantly attenuated in SKOV3 and OVCAR3 cells overexpressing SPARC. Cocultures of ovarian cancer-mesothelial cells increased the chemotactic effect on macrophages by five-fold relative to that exerted by ovarian cancer cells alone. The chemotactic activity was significantly abrogated by overexpression of SPARC in ovarian cancer cells or by the presence of an MCP-1 neutralizing antibody.

### EXAMPLE 3

This example demonstrates an inhibitory effect of SPARC on MCP-1-induced ovarian cancer cell proliferation and invasion.

The proliferation of SKOV3 and OVCAR3 transduced or not with Ad-GFP or Ad-GFP-SPARC as described in Example 1, in response to MCP-1, was assessed using a commercially available CellTiter96 kit (Promega) according to the manufacturer' instructions. The number of proliferating cells was determined colorimetrically by measuring the absorbance at 590 nm (0D590) of the dissolved formazan product after the addition of 3 -(4,5 -dimethylthiazol-2-yl)-5-(3 -carboxymethoxyphenyl)-2-(4-sulfophenyl)-2H-tetrazolium (MTS) for 3 hours.

The invasiveness of SKOV3 and OVCAR3 transduced or not with Ad-GFP or Ad-GFP-SPARC as described in Example 1, in response to MCP-1, was assessed as previously described (Said et al., Am J Pathol, 167: 1739-52 (2005)). 1×105 cells per 100 SFM in the presence and absence of MCP-lwere added to the upper chamber of polycarbonate inserts (8-mm pore size, Corning Costar, Corning, N.Y.) coated on ice with Growth factor—reduced Matrigel (BD Biosciences, Bedford, Mass.) diluted 1:3 in SFM. In some experiments, cells were pretreated with MCP-1 antibody (25 □g/ml) or its isotype control (50 μg/ml) for 30 minutes before stimulation. The bottom chambers contained complete growth medium. Assays were carried out for 6 hours at 37° C. in a 5% CO2 humidified incubator. The migrated cells were counted in six fields after staining with hemacolor 3 stain (SOURCE?), using an inverted microscope equipped with a DFC 320 digital camera (Leica Microsystems, Wetzlar, Germany) under 200× magnification.

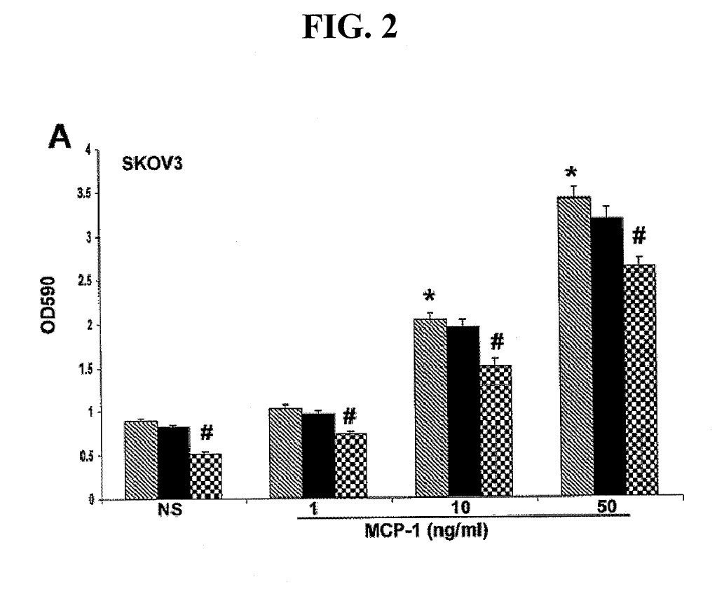

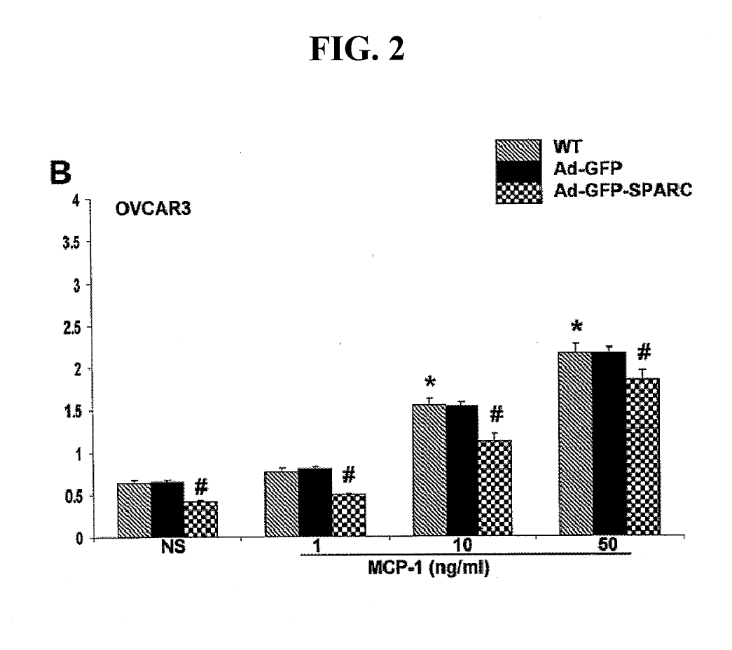

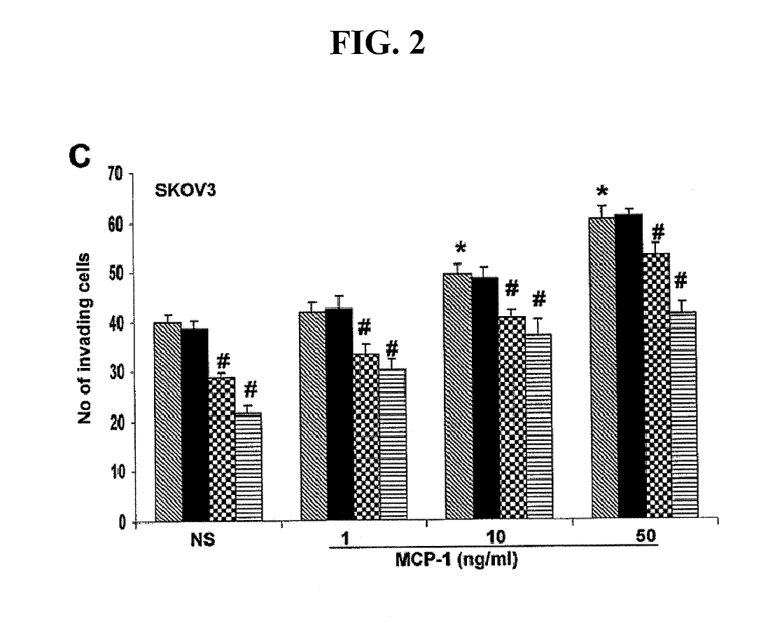

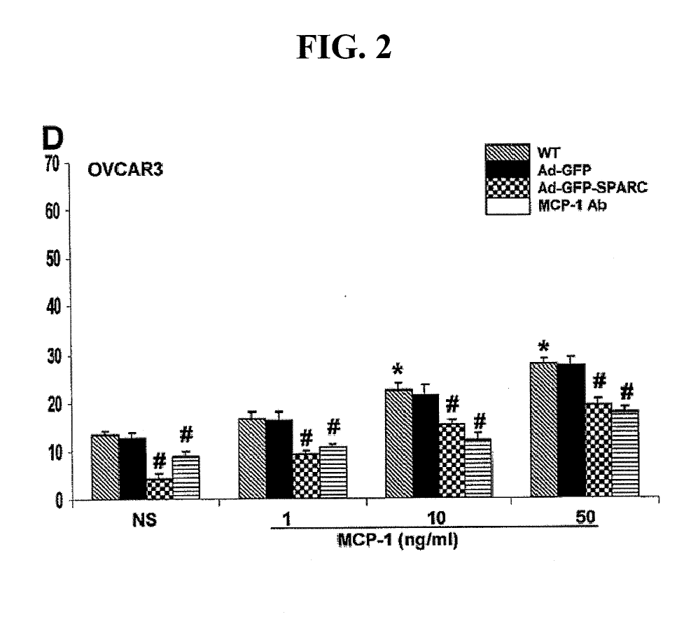

The results shown in FIG. 2 are the mean±SEM of three independent experiments performed in quadruplicate (proliferation) or triplicate (invasion). *P<0.05, compared to control NS cells. #P<0.01, compared to matched WT or Ad-GFP-transduced cells.

Overexpression of SPARC in ovarian cancer cells abrogated their responses to the mitogenic (17% to 23% and 15% to 27% inhibition for SKOV3 and OVCAR3, respectively) and the proinvasive (15% to 28% and 30% to 43% inhibition for SKOV3 and OVCAR3, respectively) effects of MCP-1. This example demonstrated that MCP-1 exerts a mitogenic and proinvasive effect on ovarian cancer cells and that expression of SPARC in ovarian cancer cells attenuates the effects of MCP-1 on proliferation and invasion.

### EXAMPLE 4

This example demonstrates an inhibitory effect of SPARC on macrophage-induced ovarian cancer cell invasion.

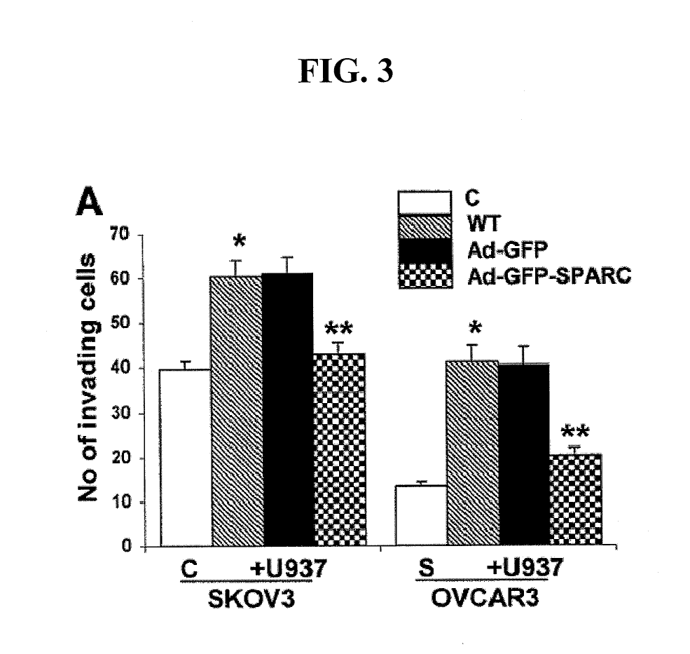

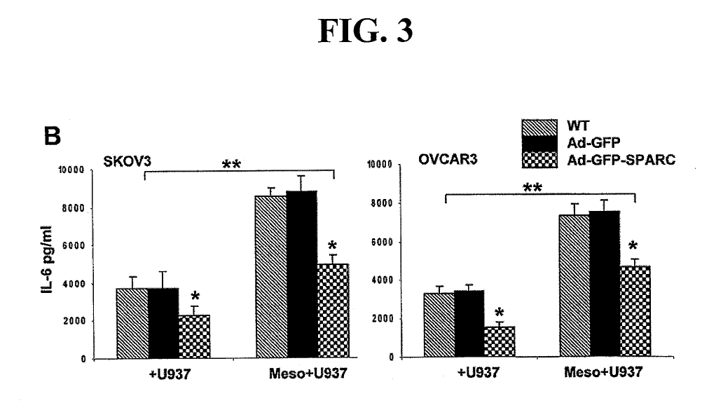

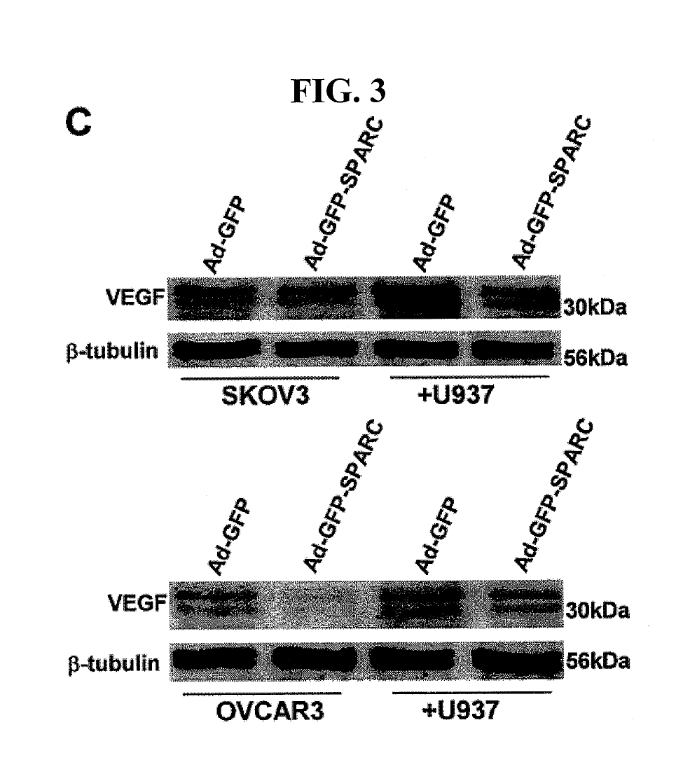

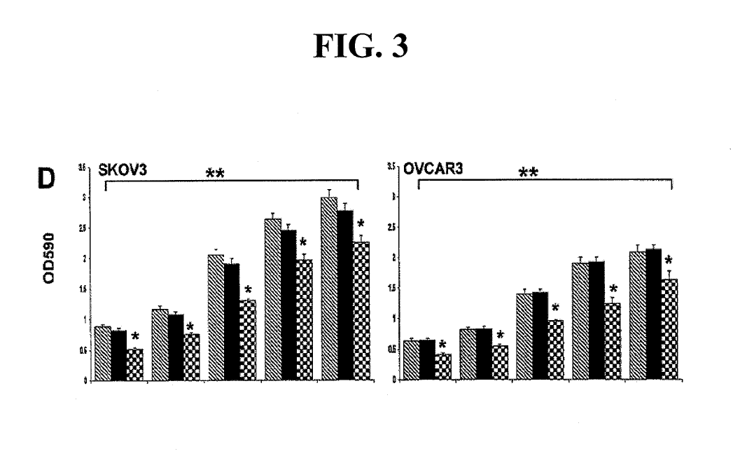

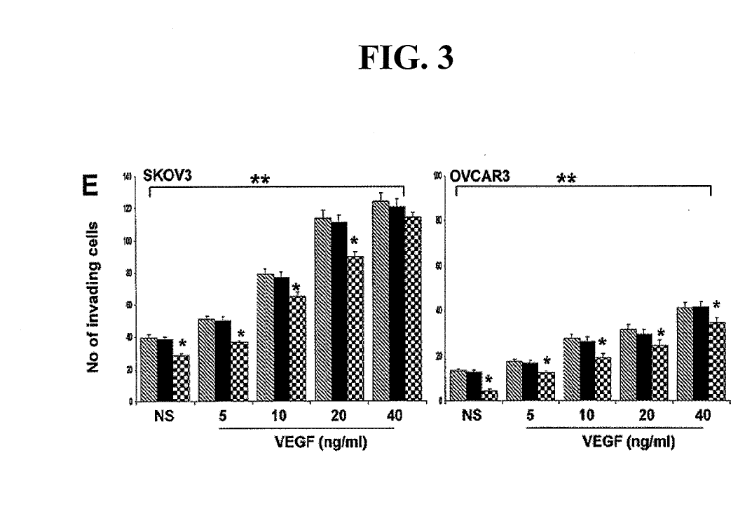

SKOV3 and OVCAR3 cells, transduced or not with Ad-GFP or Ad-GFP-SPARC as described in Example 1, were seeded into the upper chambers of Matrigel-coated inserts (10⁵ cells per 100 μl SFM per well). U937 macrophages (2×10⁶ cells per 600 μl SFM) were seeded in the lower chambers. The number of invading cells was determined as described in Example 3. The results shown in FIG. 3 are the mean±SEM of three independent experiments performed in triplicate. *P<0.05, compared to control SKOV3 and OVCAR3 cells in single-cell cultures using complete growth medium (CGM) in the bottom chamber.

Cocultures of U937 macrophages with tumor cells significantly increased the invasiveness of SKOV3 (−50%) and OVCAR3 (approximately three-fold) cell lines over single-cell cultures. Expression of SPARC in the ovarian cancer cell lines significantly abrogated the macrophage-induced invasiveness of SKOV3 and OVCAR3 by 48% and 50%, respectively.

This example demonstrated that macrophages exert a stimulatory effect on ovarian cancer cell invasion, which effect is attenuated by the expression of SPARC in ovarian cancer cells.

### EXAMPLE 5

This example demonstrates that SPARC inhibits IL-6 production in ovarian cancer cells cocultures with macrophages or with macrophages and mesothelial cells.

SKOV3 and OVCAR3 cells, transduced or not with Ad-GFP or Ad-GFP-SPARC, and U937 cells were cultured individually, in cocultures, or in triple cultures with Meso301 cells for twenty four hours, and the conditioned medium was assayed for IL-6 by ELISA as described in Example 1. The results shown in FIG. 3 represent the mean±SEM of one of three independent experiments, each performed in duplicate. *P<0.05, compared to matched WT or Ad-GFP. **P<0.05, compared to double cultures in the absence of mesothelial cells.

In cocultures of ovarian cancer cells and macrophages, IL-6 levels were significantly augmented over the basal secretion by SKOV3 (17-fold), OVCAR3 (17.6-fold), or U937 (25- to 28-fold). Restoring SPARC expression in ovarian cancer cells significantly decreased IL-6 production in cocultures with SKOV3 and OVCAR3 by 39% and 55%, respectively. Incorporating Meso301 in the triple cultures resulted in a significant increase in IL-6 production (2.5-fold over U937-SKOV3 and U937 OVCAR3 cocultures). This increase was attenuated by restoring SPARC expression in SKOV3 and OVCAR3 cells (44% and 28%, respectively). Neither exogenous SPARC nor overexpression of SPARC in U937 had any effect on the IL-6 levels. This example demonstrated that IL-6 secretion by ovarian cancer cells is augmented by coculture with macrophages and triple cultures with macrophages and mesothelial cells, and that expression of SPARC in ovarian cancer cells attenuates IL-6 secretion in these cultures.

### EXAMPLE 6

This example demonstrates an inhibitory effect of SPARC on the proteolytic activity of ovarian cancer cells and macrophages.

Subconfluent monolayers of ovarian cancer cells, transduced or not with Ad-GFP and Ad-GFP-SPARC, were grown in six-well plates alone or in coculture with mesothelial cell monolayers and serum-starved in SFM overnight. U937 macrophages (2×106 cells/ml SFM) were cultured in 0.22-μm transwell chambers (Corning, Costar) alone, in cocultures without direct cell-cell contact with ovarian cancer cells, or in triple cultures with ovarian cancer cells and mesothelial cells for an additional 24 hours. LPA (50 μM) or exogenous SPARC (20 μg/ml) (SOURCE?) was included in some experiments. Cell culture conditioned media (CM) were collected, cleared by centrifugation, and concentrated five-fold using Centricon centrifugal filters (Millipore, Bedford, Mass.). Cells in upper and lower chambers were harvested in lysis buffer (20 mM Tris, pH 7.4, 150 mM NaCl, 1 mM EDTA, 50 mM NaF, 0.5% sodium deoxycholate, 1% NP-40, 1 mM Na3VO4, and 1× protease inhibitor cocktail mixture). Lysates were cleared by centrifugation at 12,000g for 20 minutes at 4° C., and protein concentrations were determined by BCA assay (Pierce, Rockford, Ill.). CM equivalent to 200 μg of ovarian cancer cells, and/or U937 cells was analyzed by gelatin zymography as described previously (Said et al., Am J Pathol, 167: 1739-52 (2005)). Cell lysates (50 μg) of SKOV3, OVCAR3, and/or U937 cells were resolved by 10% SDS-PAGE, transferred onto polyvinylidene fluoride membranes (BioRad, Hercules, Calif.), and probed with a monoclonal antibody against β-tubulin to ensure equal protein loading. Protein detection was carried out using HRP-conjugated secondary antibodies and a SuperSignal West Dura Chemiluminescence kit (Pierce, Rockford, Ill.).

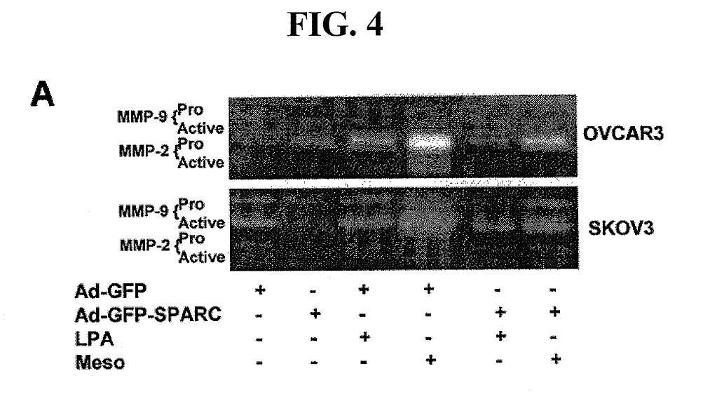

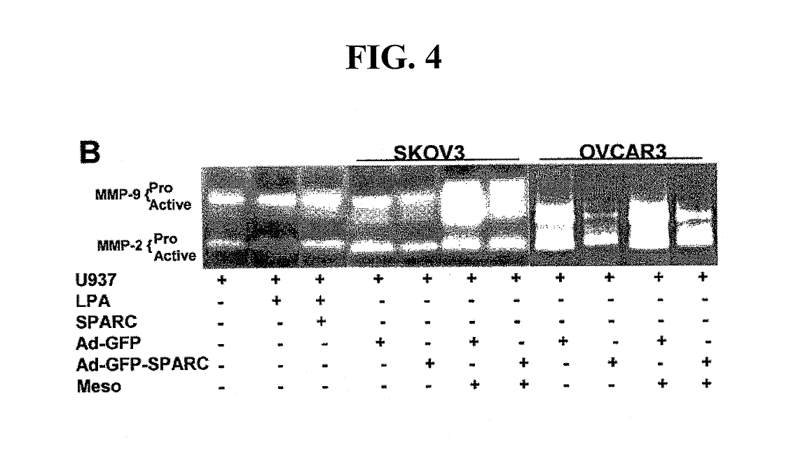

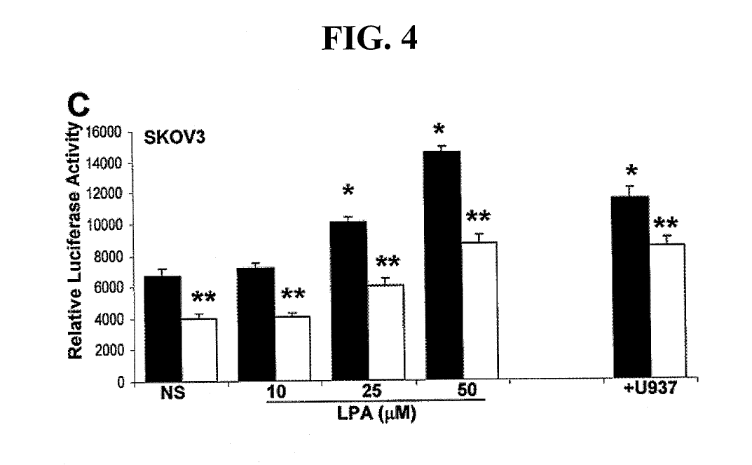

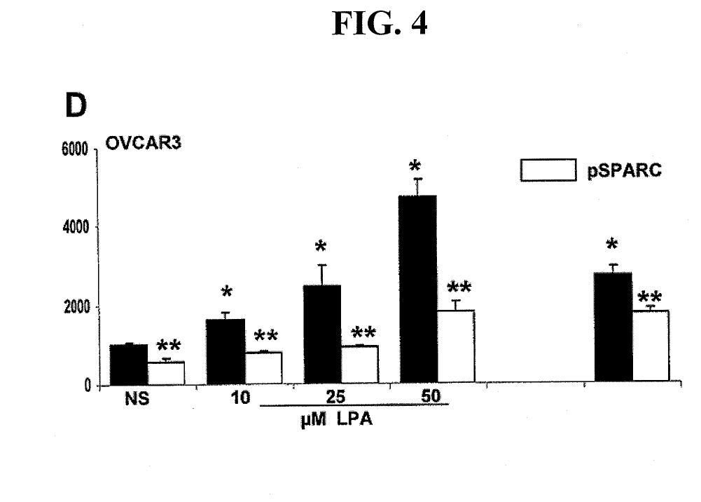

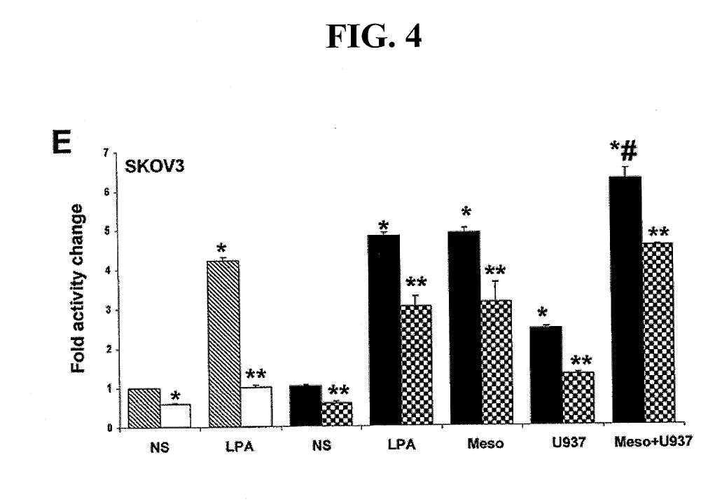

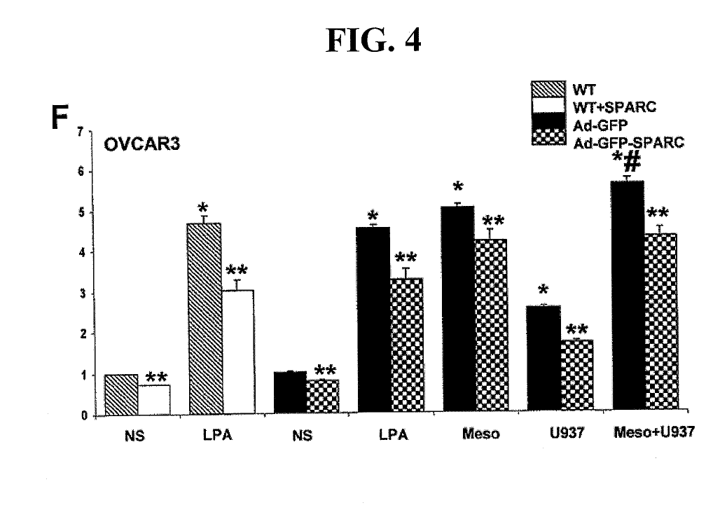

The results shown in FIG. 4 demonstrate that whereas CM of OVCAR3 had barely detectable basal activity of pro- and active MMP-9, that of SKOV3 showed a significant activity of pro- and active MMP-9 as well as pro-MMP-2. There was no difference in the enzymatic activity of these gelatinases between WT and Ad-GFP-tranduced ovarian cancer cells. Ad-GFPSPARC transduction decreased the levels of pro- and active MMP-9 in OVCAR3, whereas in SKOV3, it decreased both MMP-2 and MMP-9 activity without affecting either pro-MMP-2 or MMP-9 levels. LPA stimulation significantly increased the levels of active MMP-9 in OVCAR3 and that of pro- and active MMP-9 in SKOV3. Ad-GFP-SPARC transduction markedly decreased LPA-induced MMP-9 activity in both OVCAR3 and SKOV3. Coculture of either ovarian cancer cell line with Meso301 resulted in the most significant increase in the levels and activity of the pro and active forms of MMP-2 and MMP-9.

The CM of U937 exhibited the highest basal activity level of MMP-2 and MMP-9 compared with either SKOV3 or OVCAR3 (FIG. 4). LPA significantly increased MMP-2 and MMP-9 activity. Exogenous SPARC (20 μg/ml) markedly decreased LPA-induced activity of both MMP-2 and MMP-9. Coculture of U937 with either SKOV3 or OVCAR3 resulted in a marked increase in the levels of pro- and active MMP-9 and MMP-2. Both were significantly attenuated by Ad-SPARC transduction of ovarian cancer cells. CM from triple cultures including Meso301 cells exhibited a pronounced increase in the levels and activity of pro- and active MMP-2 and MMP-9. The inhibitory effect of Ad-SPARC transduction was more pronounced on MMP-9 levels and activity than on MMP-2 in SKOV3 and OVCAR3.

This example demonstrated that that the proteolytic activity of MMP-2 and MMP-9 in ovarian cancer is attributed not only to the constitutive production by ovarian cancer cells and macrophages, but also to their activation by interaction of ovarian cancer cells with mesothelial cells and macrophages. This example further demonstrated that these interactions differentially regulate the levels and activity of both MMP-2 and MMP-9 in SKOV3, OVCAR3, and U937 macrophages, and that changes in MMP-9 are more pronounced both in response to the stimulatory effects of LPA in cocultures and to the inhibitory effect of SPARC adenoviral gene transfer. The pattern of induction of MMP-2 and MMP-9 activity and their inhibition by Ad-SPARC indicates a possible role for SPARC in downregulation of the activity levels of these MMPs.

### EXAMPLE 7

This example demonstrates that SPARC attenuates urokinase-type plasminogen activator (uPA) activity.

SPARC plasmid (pSPARC) was prepated by cloning the human SPARC open reading frame under the control of the cytomegalovirus promoter into pcDNA3.1 (Invitrogen, Carlsbad, Calif.) using the TOPO sites. A plasmid of the uPA promoter ligated to a luciferase reporter and a β-galactosidase reference plasmid were well known in the art (uPA: Dr. Shuang Huang (Medical College of Georgia, Augusta, Ga.)). Ovarian cancer cell lines (5×104/well of 24-well plate) were cotransfected with 1.5 □g uPA promoter luciferase reporter plasmid, 0.5 μg pSPARC, and 0.2 μg of a β-galactosidase plasmid using Lipofectamine 2000 (Invitrogen, Carlsbad, Calif.) for 24 hours, and then starved for an additional 24 hours. Luciferase activity was determined after LPA (10 to 50 μM) stimulation for 6 hours or after coculture with U937 cells in 0.22-μm inserts using the commercially available Luciferase Assay Solution, Bright Glo (Promega) according to the manufacturer's instructions. β-Galactosidase activity was measured after cell lysis (Glo Lysis Buffer, Promega, Madison, Wis.) using the commercially available Galacto-Star System (Applied Biosystems, Bedford, Mass.) in order to normalize the luciferase activity and calculate relative luciferase units (RLU). The results shown in FIG. 4 are the mean±SEM of three independent experiments performed in triplicate. *P<0.05, compared to NS controls. **P<0.05, compared to matched experimental condition without pSPARC cotransfection.

Transfection of ovarian cancer cells with pSPARC significantly decreased the basal activity of the uPA promoter in both SKOV3 (42%) and OVCAR3 (˜43%). LPA stimulation of uPA promoter activity was concentration dependent in both SKOV3 (6% to 115%) and OVCAR3 (38% to 325%). Cotransfection with pSPARC significantly attenuated LPA-induced uPA promoter activity in SKOV3 (40% to 43% inhibition) and OVCAR3 (51% to 63%). Coculture of either SKOV3 or OVCAR3 cells with U937 macrophages resulted in significant stimulation of uPA promoter activity (69% and 170% increase in SKOV3 and OVCAR3, respectively), which increase was partially, although significantly, attenuated by pSPARC cotransfection (26% and 36% inhibition in SKOV3 and OVCAR3, respectively).

uPA activity in CM of confluent monolayers (˜10⁶ cells) of serum-starved SKOV3 and OVCAR3 was measured in WT cells stimulated for 24 hours with LPA (50 μM) in the presence and absence of SPARC (10 μg/ml). uPA activity in adenovirus-transduced SKOV3 and OVCAR3 stimulated or not with LPA, or cocultured with Meso301 monolayers, U937 macrophages (added in 0.22-μm transwell inserts), or in triple-culture systems was also measured. uPA activity in CM was measured using a commercially available colorimetric assay kit (Chemicon) according to the manufacturer's instructions. The results shown in FIG. 4 demonstrate uPA activity expressed as fold change from control WT SKOV3 or OVCAR3 that were not stimulated (NS), which were assigned a value of 1. The results are mean±SEM of a representative of three independent experiments, each performed in duplicate. *P<0.05, compared to nonstimulated WT or Ad-GFP cells. **P<0.05, compared to matched WT or Ad-GFP condition. #P<0.05, compared to coculture of ovarian cancer cells with either Meso301 or U937 in two-cell culture systems.

The constitutive uPA activity in CM of SKOV3 and OVCAR3 was significantly attenuated by either exogenous SPARC or Ad-SPARC transduction (40% and 29% in SKOV3 and OVCAR3, respectively). Exogenous SPARC significantly inhibited LPA-induced uPA activity in SKOV3 (76%) and OVCAR3 (36%). LPA induced a 4- to 4.5-fold increase in uPA activity in untransduced WT and Ad-GFP-transduced SKOV3 and OVCAR3 controls, respectively, which was significantly inhibited by Ad-SPARC transduction (38% and 28% inhibition, for SKOV3 and OVCAR3, respectively). Coculture of either SKOV3 or OVCAR3 with Meso301 resulted in an increase in uPA activity in their CM, comparable to that of LPA stimulation. Ad-SPARC transduction resulted in a significant decrease in uPA activity by 160% and 60% in SKOV3 and OVCAR3, respectively. Furthermore, cocultures of U937 with either SKOV3 or OVCAR3 increased uPA activity in CM by 1.7- to 2-fold that was attenuated by Ad-SPARC transduction of SKOV3 (110%) and OVCAR3 (70%). Moreover, triple cultures, including Meso301 with ovarian cancer cells and U937 macrophages, resulted in a significantly augmented uPA activity in their CM (6.2- and 5.6-fold for SKOV3 and OVCAR3, respectively) and was shown to be attenuated by Ad-SPARC transduction in SKOV3 (160%) and OVCAR3 (120%). This example demonstrated that SPARC inhibits LPA-induced uPA activity in ovarian cancer cells.

### EXAMPLE 8

This example demonstrates that SPARC attenuates PGE2 production and activity in ovarian cancer cells cultured alone or in cocultures with mesothelial cells and/or macrophages.

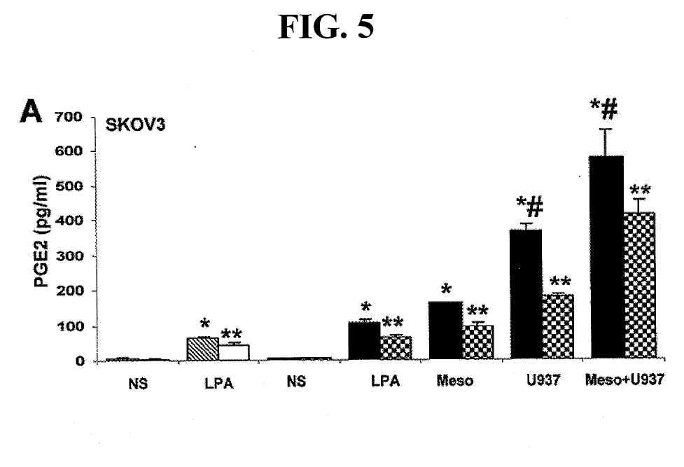

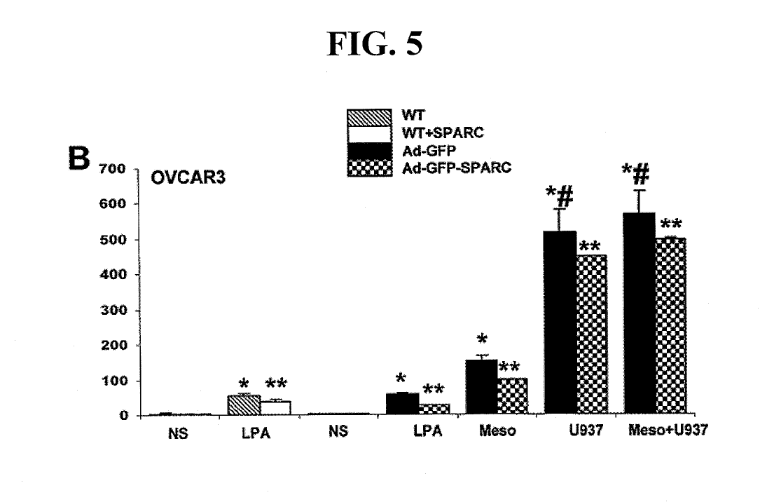

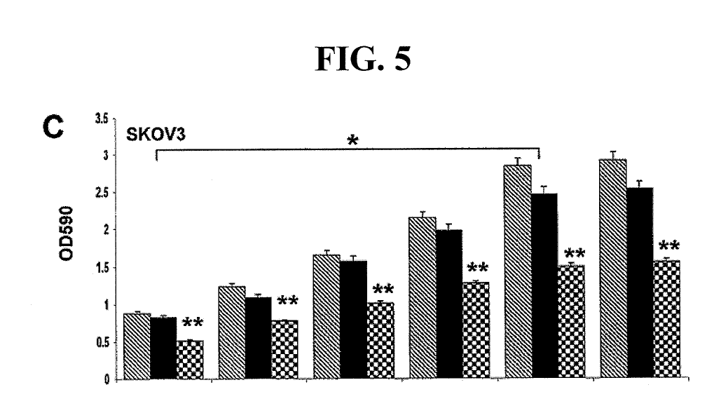

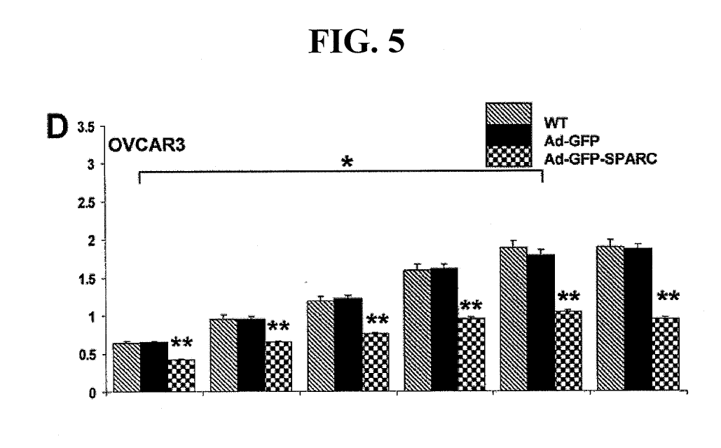

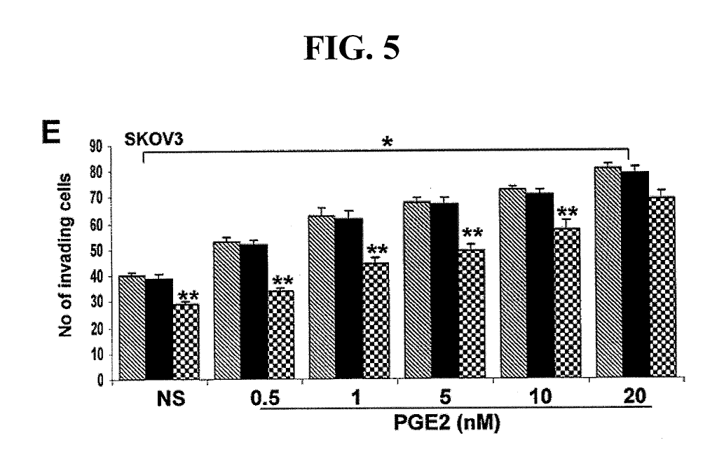

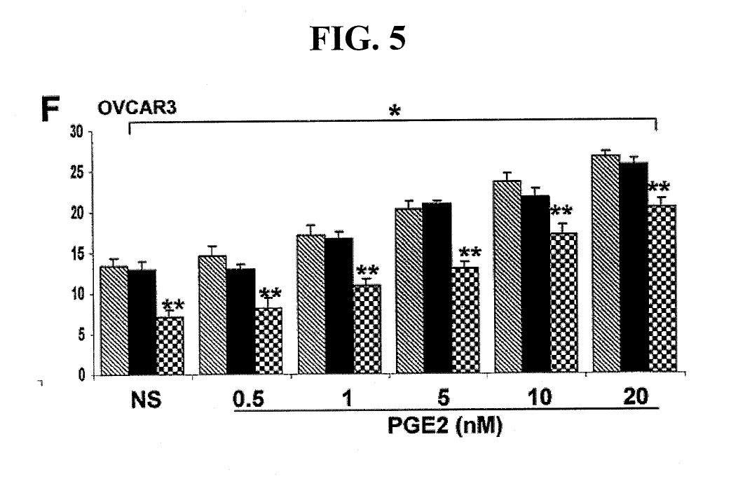

SKOV3 and OVCAR3 cells were treated as described in Example 6, and PGE2 level was determined in the CM using a commercially available enzyme immunoassay kit (Cayman Inc.) according to the manufacturer's instructions. The results are shown in FIG. 5. *P<0.05, compared to nonstimulated WT or Ad-GFP cells. **P<0.05, compared to matched WT or Ad-GFP condition. #P<0.05, compared to cocultures of ovarian cancer cells with either Meso301 or U937 in a coculture system.

Baseline production of PGE2 by serum-starved SKOV3 (−7 pg/ml) and OVCAR3 (˜5 pg/ml) was decreased by both exogenous and Ad-SPARC transduction by up to 30% to 50%, respectively. LPA stimulation of SKOV3 and OVCAR resulted in -14-fold increase in PGE2 production, which was suppressed 40% to 55% by exogenous or Ad-SPARC transduction of either cell line, respectively. PGE2 production was augmented to 22- to 30-fold by coculture of Meso301 with SKOV3 and OVCAR3, respectively. Transduction of SKOV3 and OVCAR3 with Ad-SPARC resulted in significant (40%) decrease in PGE2 production. Similar augmentation of PGE2 production was observed in cocultures of SKOV3 or OVCAR3 with U937 macrophages. Ad-SPARC transduction resulted in a significant decrease in PGE2 production in cocultures involving SKOV3 (50%), but not with OVCAR3 (14%). Further amplification of PGE2 production (80- to 100-fold) was observed in triple cultures and was significantly attenuated by Ad-SPARC transduction of SKOV3 but not of OVCAR3.

The proliferation and invasiveness of SKOV3 and OVCAR3 cells, transduced or not with Ad-GFP and Ad-GFP-SPARC, in response to PGE2, was determined as described in Example 3. The results shown in FIG. 5 are expressed as the mean±SEM of three independent experiments performed in quadruplicate (proliferation) or triplicate (invasion). *P<0.05, compared to the matched WT cells or Ad-GFP transduced cells. **P<0.05, compared to NS control cells.

PGE2-stimulated proliferation of SKOV3 and OVCAR3 was concentration dependent. Ad-SPARC transduction significantly attenuated the response of SKOV3 and OVCAR3 to PGE2-induced proliferation by 28% to 38% in SKOV3 and 32% to 49% in OVCAR3, respectively. The proinvasive effect of PGE2 on SKOV3 and OVCAR3 was concentration dependent and was significantly inhibited by Ad-SPARC (12% to 35% and 20% to 45% in SKOV3 and OVCAR3, respectively). This example demonstrated that SPARC attenuates PGE2 production and activity in ovarian cancer cells cultured alone or in cocultures with mesothelial cells and/or macrophages.

### EXAMPLE 9

This example demonstrates an inhibitory effect of SPARC on 8-isoprostane production in ovarian cancer cells cultured alone or in cocultures with mesothelial cells and/or macrophages.

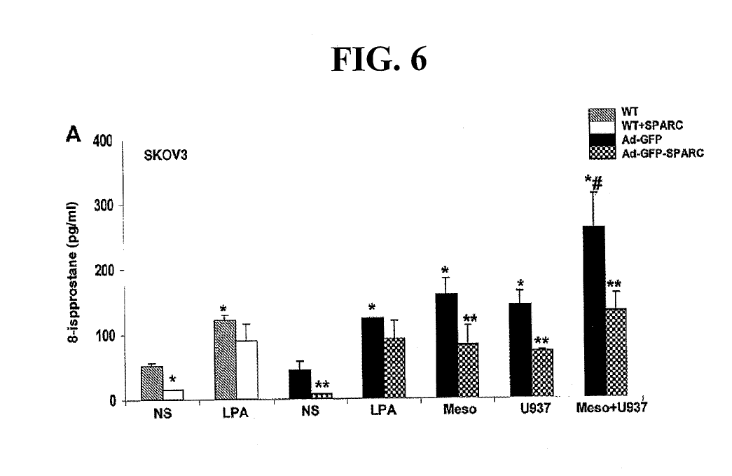

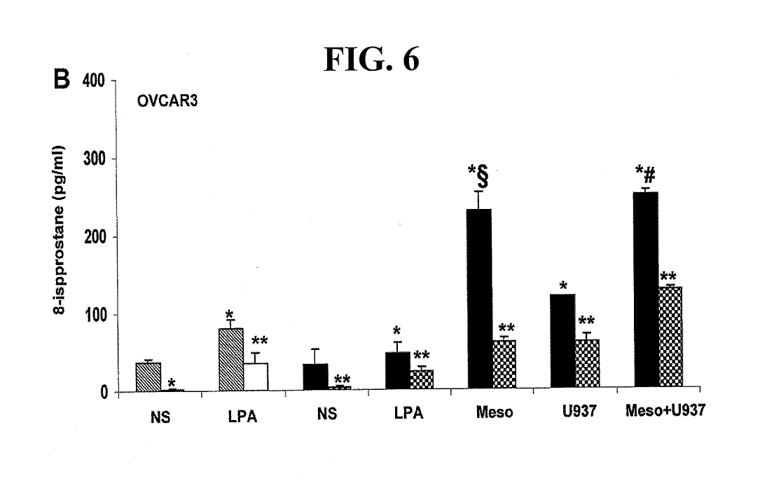

SKOV3 and OVCAR3 were treated as described in Example 6, and the level of 8-isoprostane was determined in the CM using a commercially available enzyme immunoassay kit (Cayman Inc.) according to the manufacturer's instructions. The results are shown in FIG. 6. *P<0.05, compared to nonstimulated WT or Ad-GFP cells. **P<0.05, compared to matched WT or Ad-GFP condition. #P<0.05, compared to coculture of ovarian cancer cells with either Meso301 or U937 in two-cell culture systems. §P<0.05, between cocultures of OVCAR3-Meso and OVCAR3-U937.

It has been shown that increased production of 8-isoprostane in murine peritoneal ovarian carcinomatosis is positively correlated with enhanced tumor growth, increased tumor-associated macrophages and tumor-infiltrating macrophages, as well as augmented levels of mitogenic and inflammatory cytokines and growth factors in SPARC knockout mice compared with controls (Said et al., Mol Cancer Res, 5: 1015-30 (2007)). The results in FIG. 6 show that the basal levels of reactive oxygen species (ROS) production, as measured by 8-isoprostane in serum starved SKOV3 (52 pg/ml) and OVCAR3 (38 pg/ml), was decreased by both exogenous SPARC and Ad-SPARC by 70% to 90%, respectively. LPA stimulation of SKOV3 and OVCAR resulted in an increase (˜2.2- to 2.5-fold, respectively) in 8-isoprostane production, which was suppressed up to 26% to 55% by addition of exogenous SPARC or Ad-SPARC transduction of either cell line, respectively. The production of 8-isoprostane was amplified to approximately seven- and four-fold in cocultures of Meso301 with SKOV3 and OVCAR, respectively. Transduction of SKOV3 and OVCAR3 with Ad-SPARC resulted in a significant inhibition of ROS by 48% and 74% in cocultures of Meso301 with SKOV3 and OVCAR3, respectively. Similarly, cocultures of SKOV3 or OVCAR3 with U937 macrophages resulted in amplification of 8-isoprostane production that was significantly decreased (−50%) by Ad-SPARC transduction of either ovarian cancer cell line. Further amplification of 8-isoprostane production (five- to eight-fold) was observed in triple cultures and was significantly attenuated by Ad-SPARC transduction of SKOV3 (45%) and OVCAR3 (52%). This example demonstrated that SPARC inhibits 8-isoprostane production in ovarian cancer cells cultured alone or in cocultures with mesothelial cells and/or macrophages.

### EXAMPLE 10

This example demonstrates an inhibitory effect of SPARC on the activity of the NF-kB promoter.

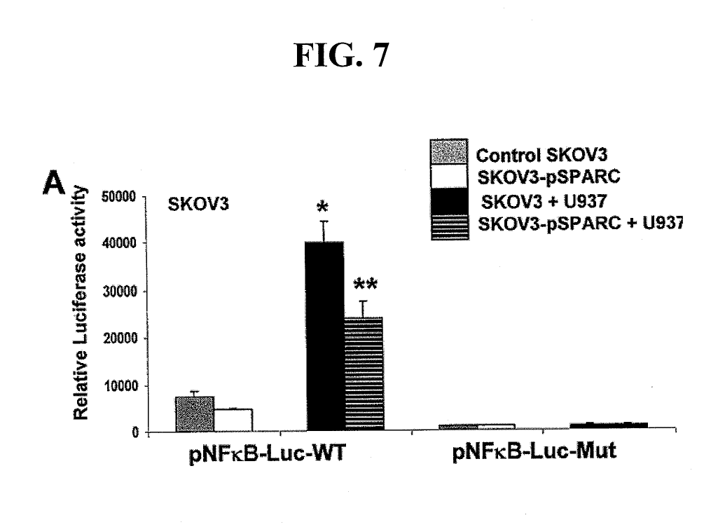

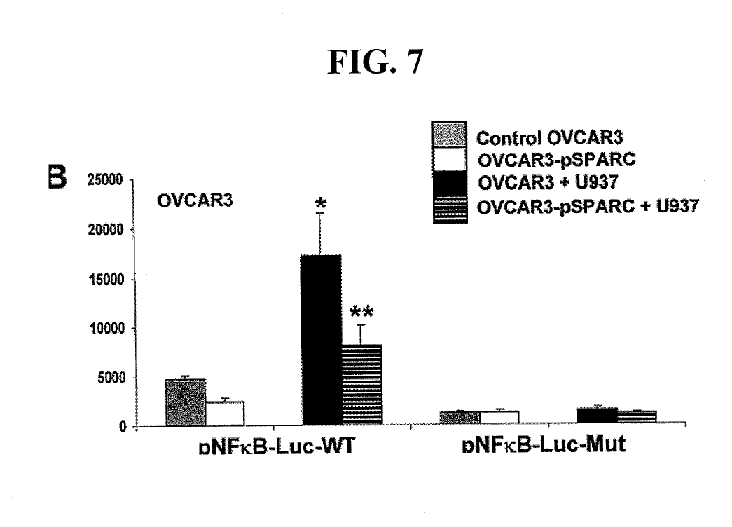

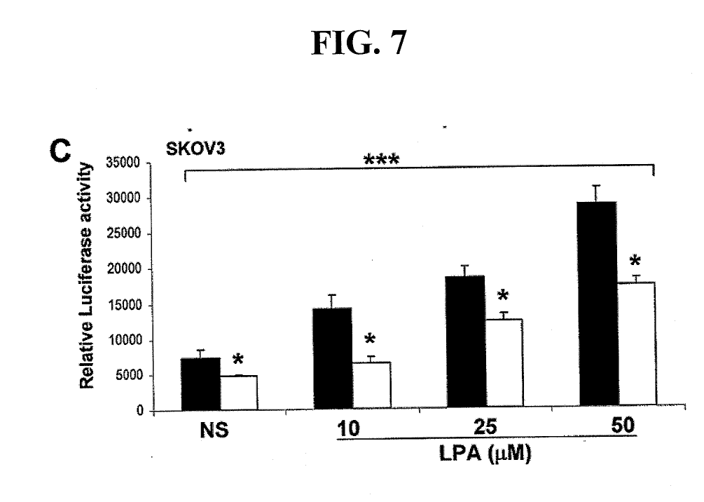

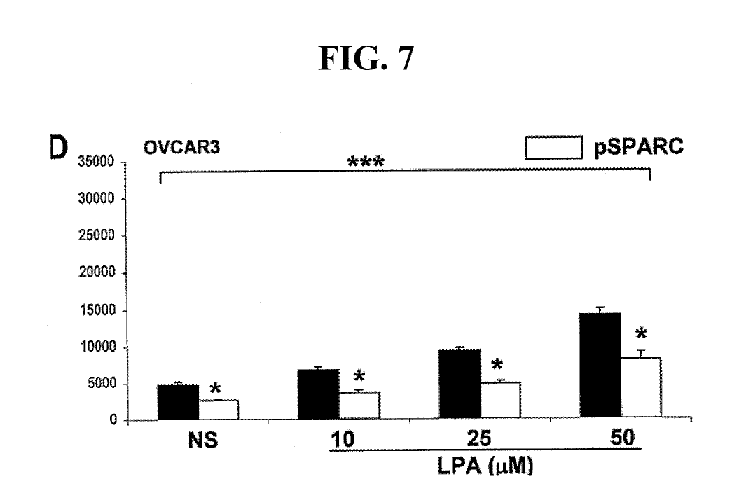

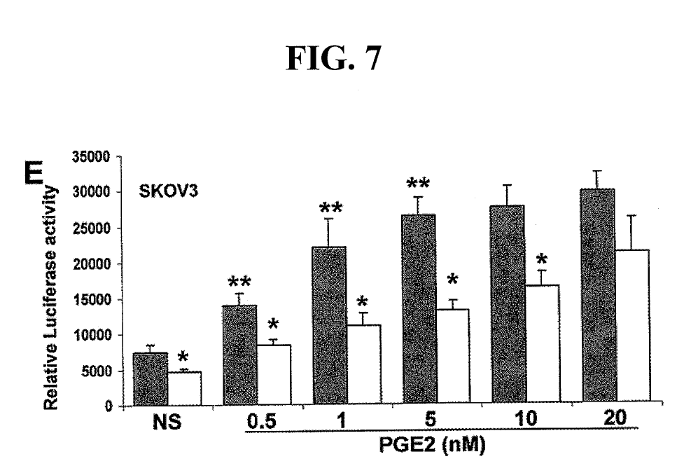

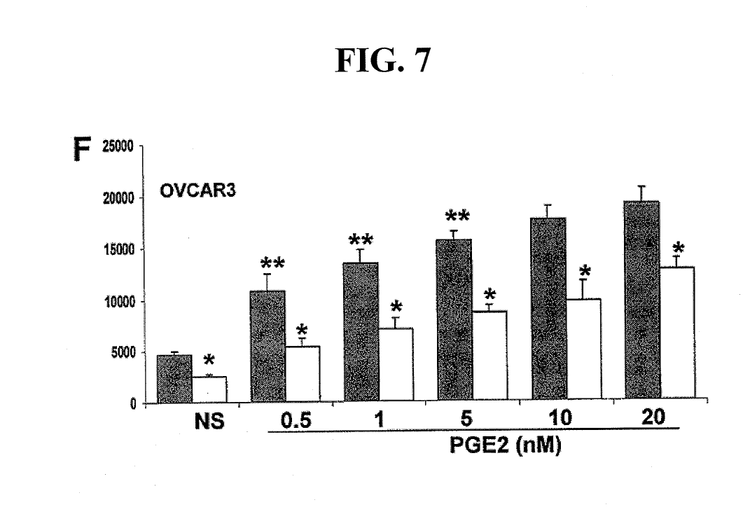

SKOV3 and OVCAR3 were transfected with 0.5 μg pSPARC, either a plasmid of NF-κB that contained two copies of the wild-type NF-κB (WT-pNF-κB-Luc) or the mutated NF-κB (Mut-pNF-κB-Luc) binding sites ligated to a luciferase reporter (NF-κB: kindly provided by Dr. Jinsong Liu (The University of Texas M. D. Anderson Cancer Center, Houston, Tex.), and 0.2 μg of a β-galactosidase plasmid, and were cocultured with U937 without direct cell-cell contact, or stimulated with LPA or PGE2. Relative luciferase activity was determined as described in Example 7. The results shown in FIG. 7 represent the mean±SEM of the relative luciferase activity, corrected to β-gal activity, of three independent experiments performed in triplicates. *P<0.05, compared to SKOV3 and OVCAR3 in single-cell culture, or compared to matched NS and LPA- or PGE2-stimulated cells without pSPARC cotransfection. **P<0.05, between cocultures without pSPARC transfection of ovarian cancer cells. **P<0.05, compared to NS and between the tested concentrations of PGE2. ***P<0.05, compared to NS, and between each concentration of LPA stimulation.

The activation of NF-κB, which is seen in most cancer cells, plays a key role in tumor initiation, progression, metastasis, and chemoresistance by mediating the production of a large variety of proinflammatory cytokines, chemokines, growth factors, collagenases, and antiapoptotic proteins. The results shown in FIG. 7 show that cocultures of U937 macrophages with SKOV3 and OVCAR3 cells resulted in a significant increase in NF-κB activation compared with non-cocultured ovarian cancer cells (5- and 3.5-fold increase in SKOV3 and OVCAR3, respectively). Transient transfection of ovarian cancer cells with pSPARC attenuated the macrophage-induced NF-κB activation by 40% and 53% in SKOV3 and OVACR3, respectively. LPA-induced NF-κB activation (40% to 50%) in both SKOV3 and OVCAR3. Cotransfection of SKOV3 and OVCAR3 with pSPARC attenuated LPA-induced NF-κB activation by ˜50% in both cell lines. PGE2 treatment of SKOV3 and OVCAR3, resulted in a concentration-dependent NF-κB activation (approximately three-fold) up to 5 nM. Cotransfection of ovarian cancer cells with pSPARC resulted in a significant (30% to 50%) attenuation of PGE2-induced NF-κB activation in both cell lines. Transient transfection of either SKOV3 or OVCAR3 with pSPARC significantly decreased (40%) their basal activity of NF-κB. This example demonstrated that SPARC attenuates the activity of the NF-κB promoter in ovarian cancer cells.

### EXAMPLE 11

This example demonstrates that SPARC negatively regulates the expression of pro-inflammatory genes.

Human ovarian carcinoma cells (OVCAR3) stably encoding a control green fluorescent protein (GFP) or SPARC-GFP were used. 2-4×10⁶ cells were implanted into the peritoneal cavity of nude mice and tumor development was monitored for 4 weeks. Total RNA was isolated from solid tumors, and cDNA was prepared for microarray analysis using Agilent Human Whole Genome 4×44K single color GE chips.

Overexpression of SPARC in intraperitoneally-implanted ovarian carcinoma cells resulted in the downregulation of several inflammation pathways, including the interleukin (IL) IL-1, the IL-2/IL-4, and the TNF-α pathways. Examples of genes in the IL-1 pathway whose expression was significantly changed in intraperitoneal tumors expressing SPARC-GFP relative to those expressing the control GFP include IL-1β (downregulated 2.57-fold) and the IL-1 receptor (downregulated 1.82-fold). MAP3K7, also known as TGF-β-activated kinase 1 or TAK1, which plays an essential role in the innate and adaptive immune responses, was downregulated 7.44-fold. Pellinol, also known as PELI1, which is required for NF-κB activation and IL-8 expression in response to IL-1, was downregulated 6.09-fold. The IL-1 receptor accessory protein, also known as IL1RAP, which contributes to the antagonism of IL-1 action, was upregulated 3.48-fold.

Several genes in the IL-2/IL-4 pathway were significantly changed in intraperitoneal tumors expressing SPARC-GFP relative to those expressing the control GFP. ETS-1, which is a critical regulator of vascular inflammation and is essential for mounting an effective type 1 immune response, was downregulated 5.835-fold. ATF-2, which upregulates the expression of inflammation-related genes, was downregulated 5.51-fold. SOCS3, which is a suppressor of cytokine signaling, was upregulated 9.35-fold.

Several genes in the TNF-α pathway were significantly changed in intraperitoneal tumors expressing SPARC-GFP relative to those expressing the control GFP. NF-κB1B, which is a member of the IκB family of proteins that inactivate NF-κB by trapping it in the cytoplasm, was upregulated 3.53-fold. IκB kinase β, also known as IKBKB, whose anti-inflammatory function includes the inhibition of the classical pathway of macrophage activation, was upregulated 2.97-fold. GSK3 β, which plays an important role in mediating proinflammatory NF-κB activation, was downregulated 2.54-fold.

This example demonstrated that overexpression of SPARC in human ovarian carcinoma cells resulted in downregulation of the IL-1, the IL-2/IL-4, and the TNF-α pathways.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended tenns (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a mammal afflicted with an inflammatory disease or condition comprising the administration of a therapeutically effective mount of a SPARC polypeptide and a pharmacologic carrier to the afflicted mammal.

2. The method of claim 1, wherein the SPARC polypeptide is comprised of SEQ ID NO: 1.

3. The method of claim 1, wherein the inflammatory disease or condition is peritonitis, plueritis, rheumatoid arthritis, an inflammatory arthropathy, acute disseminated encephalomyelitis, Addison's disease, ankylosing spondylitis, autoimmune hepatitis, autoimmune inner ear disease, bullous pemphigoid, coeliac disease, Crohns disease, ulcerative colitis, dermatomyositis, endometriosis, Goodpasture's syndrome, Graves' disease, Guillain-Barré syndrome, Hashimoto's disease, Kawasaki disease, interstitial cystitis, lupus erythematosus, mixed connective tissue disease, multiple sclerosis, myasthenia gravis, pemphigus vulgaris, psoriasis, psoriatic arthritis, polymyositis, primary biliary chirrosis, scleroderma, sjögren's syndrome, stiff person syndrome, temporal arteritis, vasculitisatherosclerosis, rheumatoid arthritis, sepsis, acute bronchitis, pulmonary emphysema, rhinitis, sinusitis, asthma, alveolitis, farmer's lung disease, hyperreactive airways, bronchitis, pneumonitis, pediatric asthma, bronchiectases, pulmonary fibrosis, ARDS, pneumonia, interstitial pneumonitis, bronchitis, radiation induce injury, cystic fibrosis, alphal-antitrypsin deficiency, inflammatory pseudopolyps, colitis cystica profunda, pneumatosis cystoides intestinales, gallstones, renal stones, rheumatoid arthritis, psoriatic arthritis, juvenile rheumatoid arthritis, early arthritis, reactive arthritis, osteoarthritis, tendonitis, periodontal disease, fibrosis, neuroinflammation, multiple sclerosis, serositis, vitiligo, Wegener's granulomatosis, or atherosclerosis, sepsis, acute, allergic, or chronic bronchitis, chronic obstructive bronchitis, coughing, pulmonary emphysema, allergic or non-allergic rhinitis or sinusitis, chronic rhinitis or sinusitis, asthma, alveolitis, farmer's lung disease, hyperreactive airways, infectious bronchitis or pneumonitis, pediatric asthma, bronchiectases, pulmonary fibrosis, acute adult respiratory distress syndrome, bronchial edema, pulmonary edema, bronchitis, pneumonia, interstitial pneumonits, or bronchitis, pneumonia as a result of heart failure, irradiation pneumonitis, chemotherapy induced pnuemonitis, cystic fibrosis, or alphal -antitrypsin deficiency, acute cholecystitis or chronic cholecystitis.

4. The method of claim 1, further comprising the administration of an anti-inflammatory drug.

5. The method of claim 1, further comprising the administration of an antimicrobial agent.

6. The method of claim 5, wherein the antimicrobial agents is an antifungal, antiviral or antibiotic.

7. A method of treating a mammal afflicted with peritonitis comprising the intraperitoneal administration of a therapeutically effective mount of a SPARC polypeptide and a pharmacologic carrier.

8. The method of claim 7, wherein the peritonitis is a spontaneous bacterial peritonitis. 5

9. The method of claim 7, wherein the peritonitis is a chemical peritonitis.

10. The method of claim 7, wherein the peritonitis is the result of appendicitis. 7.

11. The method of claim 7, wherein the peritonitis is the result of an intestinal infract.

12. The method of claim 7, wherein the peritonitis is the result of pancreatitis.

13. The method of claim 7, wherein the peritonitis is the result of a gastric rupture.

14. The method of claim 7, wherein the peritonitis is the result of a perforating gastric ulcer.

15. The method of claim 7, wherein the peritonitis is the result of trauma.

16. The method of claim 7, further comprising the administration of an antinflammatory drug.

17. The method of claim 7, further comprising the administration of an antimicrobial agent.

18. The method of claim 7, wherein the antimicrobial agents is an antifungal, antiviral or antibiotic.

19. The method of claim 7, wherein the SPARC polypeptide is comprised of SEQ ID NO: 1.

20. A method of reducing the incidence of peritoneal adhesions in a mammal comprising the intraperitoneal administration of a therapeutically effective amount of a SPARC polypeptide and a pharmacologic carrier.

21. The method of claim 20, wherein the SPARC polypeptide is comprised of SEQ ID NO: 1.

22. The method of claim 20, wherein the peritoneal adhesions result from trauma.

23. The method of claim 20, wherein the peritoneal adhesions result from abdominal mdor pelvic surgery.

24. The method of claim 20, wherein the peritoneal adhesions result from peritonitis.

25. A method of treating a mammal afflicted with endometriosis comprising the intraperitoneal administration of a therapeutically effective amount of a SPARC polypeptide and a carrier.

26. The method of claim 25, wherein the SPARC polypeptide is comprised of SEQ ID NO: 1.

27. The method of claim 25, further comprising the administration of an anti-inflammatory drug.

28. The method of claim 25, further comprising the administration of an antimicrobial agent.

29. The method of claim 28, wherein the antimicrobial agents is an antifungal, antiviral or antibiotic.

30. A method of treating a mammal afflicted with an inflammatory disease or condition comprising the administration of a therapeutically effective mount of a SPARC polypeptide-encoding isolated polynucleotide and a pharmacologic carrier to the afflicted mammal.

31. The method of claim 30, wherein the SPARC polypeptide-encoding isolated polynucleotide comprises SEQ ID NO: 2.

32. The method of claim 30, wherein the inflammatory disease or condition is peritonitis, plueritis, rheumatoid arthritis, an inflammatory arthropathy, acute disseminated encephalomyelitis, Addison's disease, ankylosing spondylitis, autoimmune hepatitis, autoimmune inner ear disease, bullous pemphigoid, coeliac disease, Crohns disease, ulcerative colitis, dermatomyositis, endometriosis, Goodpasture's syndrome, Graves' disease, Guillain-Barré syndrome, Hashimoto's disease, Kawasaki disease, interstitial cystitis, lupus erythematosus, mixed connective tissue disease, multiple sclerosis, myasthenia gravis, pemphigus vulgaris, psoriasis, psoriatic arthritis, polymyositis, primary biliary chirrosis, scleroderma, sjögren's syndrome, stiff person syndrome, temporal arteritis, vasculitisatherosclerosis, rheumatoid arthritis, sepsis, acute bronchitis, pulmonary emphysema, rhinitis, sinusitis, asthma, alveolitis, farmer's lung disease, hyperreactive airways, bronchitis, pneumonitis, pediatric asthma, bronchiectases, pulmonary fibrosis, ARDS , pneumonia, interstitial pneumonitis, bronchitis, radiation induce injury, cystic fibrosis, alphal-antitrypsin deficiency, inflammatory pseudopolyps, colitis cystica profunda, pneumatosis cystoides intestinales, gallstones, renal stones, rheumatoid arthritis, psoriatic arthritis, juvenile rheumatoid arthritis, early arthritis, reactive arthritis, osteoarthritis, tendonitis, periodontal disease, fibrosis, neuroinflammation, multiple sclerosis, serositis, vitiligo, Wegener's granulomatosis, or atherosclerosis, sepsis, acute, allergic, or chronic bronchitis, chronic obstructive bronchitis, coughing, pulmonary emphysema, allergic or non-allergic rhinitis or sinusitis, chronic rhinitis or sinusitis, asthma, alveolitis, farmer's lung disease, hyperreactive airways, infectious bronchitis or pneumonitis, pediatric asthma, bronchiectases, pulmonary fibrosis, acute adult respiratory distress syndrome, bronchial edema, pulmonary edema, bronchitis, pneumonia, interstitial pneumonits, or bronchitis, pneumonia as a result of heart failure, irradiation pneumonitis, chemotherapy induced pnuemonitis, cystic fibrosis, or alphal-antitrypsin deficiency, acute cholecystitis or chronic cholecystitis.

33. The method of claim 30, further comprising the administration of an anti-inflammatory drug.

34. The method of claim 30, further comprising the administration of an antimicrobial agent.
